(19) United States
(12) Patent Application Publication
(10) Pub. No.: US 2023/0340068 A1
(43) Pub. Date: Oct. 26, 2023

(54) CHIMERIC ANTIGEN RECEPTOR (CAR) WITH CD28 TRANSMEMBRANE DOMAIN

(71) Applicant: The Regents of the University of California, Oakland, CA (US)

(72) Inventors: Yannick MULLER, Lausanne (CH); Jeffrey A. BLUESTONE, San Francisco, CA (US); Qizhi TANG, San Francisco, CA (US)

(21) Appl. No.: 18/044,772

(22) PCT Filed: Sep. 9, 2021
(86) PCT No.: PCT/US2021/049682

§ 371 Date: Mar. 9, 2023

Related U.S. Application Data
(60) Provisional application No. 63/077,429, filed on Sep. 11, 2020.

(51) Int. Cl. C07K 14/705; C07K 14/725; A61K 45/06; A61K 38/17; A61K 39/00; C07K 16/28

(52) U.S. Cl. CPC C07K 14/70521; C07K 14/70517; C07K 14/7051; A61K 45/06; A61K 38/1774; A61K 39/464412; A61K 39/4611; C07K 16/2803; C07K 2319/33; C07K 2319/03; C07K 2319/02; C07K 2317/53; C07K 2317/524; C07K 2317/526; C07K 2317/622; C07K 2317/76; A61K 2039/545; A61K 39/4631; A61K 2239/21; A61K 2239/22; A61K 2239/38; A61K 2239/17; A61K 2239/13

(57) ABSTRACT

The present disclosure relates generally to the field of immuno-therapeutics, and particularly relates to novel chimeric polypeptides, e.g., chimeric antigen receptors (CARs) that include a transmembrane domain from CD28 and a hinge domain. In some cases, the hinge domain is capable of promoting dimerization of the CARs. The disclosure also provides compositions and methods useful for producing such molecules, as well as methods for the detection and treatment of health conditions, such as proliferative diseases (e.g., cancer).

### CROSS-REFERENCE TO RELATED APPLICATIONS

The present application claims priority to U.S. Provisional Patent Application Ser. No. 63/077,429, filed on Sep. 11, 2020, the disclosure of which is incorporated by reference herein in its entirety, including any drawings.

### INCORPORATION OF THE SEQUENCE LISTING

The material in the accompanying Sequence Listing is hereby incorporated by reference into this application. The accompanying Sequence Listing text file, named 048536-631001WO_Sequence Listing_ST25.txt.” was created on Sep. 2, 2021 and is 16 KB.

### FIELD

The present disclosure relates generally to the field of immuno-therapeutics, and particularly relates to novel chimeric polypeptides, e.g., chimeric antigen receptors (CARs) that include a transmembrane domain from CD28 and a hinge domain. In some cases, the hinge domain is capable of promoting dimerization of the CARs. The disclosure also provides compositions and methods useful for producing such molecules, as well as methods for the detection and treatment of health conditions, such as proliferative diseases (e.g., cancer).

### BACKGROUND

In recent years, chimeric antigen receptors (CARs) have emerged as a promising approach for immunotherapy and have been evaluated in clinical trials conducted by a number of pharmaceutical and biotechnology companies. CARs are antigen-specific recombinant receptors, which, in a single molecule, redirect the specificity and function of a number of immune cells, including T lymphocytes. For example, in CAR-T cell therapy, the general premise for the use of CAR-T cells in cancer immunotherapy is to rapidly generate tumor-targeted T cells, bypassing the barriers and incremental kinetics of active immunization, and eliminating MHC restriction in antigen-recognition. Once expressed in T cells, the CAR-modified T cells acquire supra-physiological properties and act as “living drugs” that may exert both immediate and long-term effects. Multiple iterations of CARs have been developed, mainly focusing on antigen-binding moiety and intracellular signaling modules, which are deemed crucial for CAR design. For example, the FDA has approved two anti-CD19 CAR T-cell products, tisagenlecleucel (CTL019, KYMRIAH®) and axicabtagene ciloleucel (KTE-C19, YESCARTA®), for the treatment of acute lymphocytic leukemia and relapsed/refractory large B-cell lymphoma. A third CAR-T product, Lisocabtagene Maraleucel (JCAR-17, LISO-CEL), is under review by the FDA for adults with relapsed/refractory large B-cell lymphoma.

In general, to achieve appropriate costimulatory signals in order to activate effector T cells, improve response, and prolong persistence, many different types of costimulatory receptors can be incorporated, alone, in tandem, or in larger arrays. However, the effect of CAR's non-signaling modules, such as hinge and transmembrane (TM) domains, on the proliferation of the transduced T cells and therapeutic efficacy of CARs remains largely unclear. It has been reported that CAR potency is often limited, particularly in solid tumors. This is often due to low target antigen density and immune suppressive factors in the microenvironment. In addition, one of the major side effects of a number of CAR-T products is neurological and other toxicities as a consequence of cytokine storm. Insights into the knowledge of the antigen-independent signaling capability through TM domains can be used to control CAR signaling, manage off-target activity and enhance local activation at inflammatory sites in both its use in cytolytic T cells and regulatory T cells.

Consequently, there remains a need for more potent CARs to overcome these obstacles to extend the reach of these therapeutics to more diseases and to treat more patients.

### SUMMARY

The present disclosure relates generally to the development of immuno-therapeutics, such as enhanced polypeptides and chimeric antigen receptors (CARs), as well as pharmaceutical compositions comprising the same for use in treating various conditions, such as proliferative disorders (e.g., cancer). As described in greater detail below, CAR constructs containing a hinge domain and a CD28 transmembrane domain (TMD) have been found to result in surprisingly enhanced CAR functionality. In some embodiments, the hinge domain is capable of promoting dimerization of the CAR constructs. Without being bound any particular theory, it is anticipated that the some of the chimeric polypeptides and CARs describes herein represent several advantages. For example, the chimeric polypeptides and CARs describes herein can be used to reduce excessive on-target activation as they do not interact with endogenous CD28. In some embodiments, the chimeric polypeptides and CARs describes herein can be used to increase CAR-T cells survival as the wild type CD28-TMD is associated with reduced CD28 expression. In some embodiments, the chimeric polypeptides and CARs describes herein can be used to reduce CAR-T cells exhaustion as they restore CD28 expression on CAR-T cells. In some embodiments, the chimeric polypeptides and CARs describes herein can be used to reduce CAR-T cells toxicities as they are shown to be free of CD28 interactions. Furthermore, it is also contemplated that chimeric polypeptides and CARs describes herein can be used for the development of a new class of engineered CAR constructs that are functional in a monomeric state, e.g., CARs that can still be capable of dimerization, but exhibit a lack of capability or a reduced capability to form heterodimers with endogenous molecules that can affect CAR activation and function.

In one aspect, provided herein are chimeric polypeptides including: (a) an extracellular domain (ECD) having a binding affinity for an antigen; (b) a hinge domain; (c) a transmembrane domain (TMD) derived from CD28; and (d) an intracellular signaling domain (ICD).

Non-limiting exemplary embodiments of the disclosed chimeric polypeptides of the disclosure include one or more of the following features. In some embodiments, the CD28-TMD is a mouse CD28-TMD or a human CD28-TMD. In some embodiments, the TMD includes one or more amino acid substitutions within a transmembrane dimerization motif of the CD28-TMD. In some embodiments, the TMD includes an amino acid sequence having at least 70% sequence identity to a CD28-TMD having the sequence of SEQ ID NO: 1, and further includes one or more amino acid substitutions at a position corresponding to an amino acid residue selected from the group consisting of X13, X14, X15, and X19 of SEQ ID NO: 1. In some embodiments, the TMD includes the sequence of SEQ ID NO: 1, and further includes one or more amino acid substitutions at an amino acid residue selected from the group consisting of C13, Y14, S15, and T19 of SEQ ID NO: 1. In some embodiments, the TMD includes the sequence of SEQ ID NO: 1, and further includes the following amino acid substitutions: C13L, Y14L, S15L, and T19L. In some embodiments, the TMD includes the sequence of SEQ ID NO: 6, and wherein one, two, three, four, or five amino acid residues in the sequence of SEQ ID NO: 6 is optionally substituted by a different amino acid residue. In some embodiments, the TMD includes the sequence of SEQ ID NO: 6. In some embodiments, the one or more amino acid substitutions is independently selected from the group consisting of a leucine substitution, an alanine substitution, an arginine substitution, an aspartic acid substitution, a histidine substitution, a glutamic acid substitution, a lysine substitution, a serine substitution, a tryptophan substitution, and combinations of any thereof.

In some embodiments of the disclosure, the chimeric polypeptide is a chimeric antigen receptor (CAR). In some embodiments, the hinge domain capable of promoting dimerization of the chimeric polypeptide. In some embodiments, the hinge domain is derived from a CD8 hinge domain, a CD28 hinge domain, an IgG4 hinge domain, and an Ig4 CH2-CH3 domain. In some embodiments, the ICD includes one or more costimulatory domains selected from the group consisting of costimulatory domains derived from 4-1BB (CD137), CD27 (TNFRSF7), CD28, CD70, LFA-2 (CD2), CD5, ICAM-1 (CD54), ICOS, LFA-1 (CD11a/CD18), DAP10, and DAP12. In some embodiments, the ICD includes two costimulatory domains. In some embodiments, the ICD further includes one or more CD3 polypeptide chains. In some embodiments, the one or more CD3 chains includes a CD3ζ domain or a functional variant thereof.

In some embodiments, the ECD includes an antigen-binding moiety capable of binding to an antigen on the surface of a cell. In some embodiments, the antigen-binding moiety is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment of any thereof. In some embodiments, the antigen-binding moiety includes a scFv. In some embodiments, the antigen is a tumor associated-antigen or a tumor-specific antigen. In some embodiments, the antigen is selected from the group consisting of CD19 and HLA-A2.

In one aspect, provided herein are nucleic acid constructs that include a nucleic acid sequence encoding a chimeric polypeptide as disclosed herein. In some embodiments, the nucleotide sequence is incorporated into an expression cassette or an expression vector. In some embodiments, the expression vector is a viral vector. In some embodiments, the viral vector is a lentiviral vector, an adeno virus vector, an adeno-associated virus vector, or a retroviral vector. In some embodiments, the viral vector is a lentiviral vector.

In another aspect, provided herein are recombinant cells including: (a) a chimeric polypeptide of the disclosure; and/or (b) a nucleic acid of the disclosure. Also provided, in a related aspect, are cell cultures including at least one recombinant cell as disclosed herein and a culture medium. In some embodiments, the recombinant cell is a eukaryotic cell. In some embodiments, the recombinant cell is an immune system cell. In some embodiments, the immune system cell is a T lymphocyte.

In another aspect, provided herein are pharmaceutical compositions including a pharmaceutically acceptable carrier and one or more of the following: (a) a chimeric polypeptide of the disclosure; (b) a nucleic acid construct of the disclosure; and (c) a recombinant cell of the disclosure. In some embodiments, the composition includes a recombinant cell of the disclosure, and a pharmaceutically acceptable carrier. In some embodiments, the composition includes a nucleic acid construct of the disclosure, and a pharmaceutically acceptable carrier. In some embodiments, the nucleic acid construct is encapsulated in a viral capsid or a lipid nanoparticle.

In one aspect, provided herein are methods for modulating T-cell activation in a subject having or suspected of having a health condition, the methods include administering to the subject a composition including at least one recombinant cell of the disclosure; and/or a pharmaceutical composition of the disclosure.

In another aspect, provided herein are method for treating a health condition in a subject in need thereof, the methods include administering to the subject a composition including at least one recombinant cell of the disclosure; and/or a pharmaceutical composition of the disclosure. In some embodiments, the health condition is a proliferative disorder, an autoimmune disorder, or an infection. In some embodiments, the proliferative disorder is a cancer. In some embodiments, the cancer is selected from the group consisting of a lymphoma, acute lymphocytic leukemia, and relapsed/refractory large B-cell lymphoma. In some embodiments, the lymphoma is a Burkitt lymphoma.

In some embodiments of the methods disclosed herein, the administered composition results in reduced on-target activation in the subject. In some embodiments, the administered composition increases CAR-T cell survival in the subject. In some embodiments, the administered composition reduces CAR-T cell exhaustion in the subject. In some embodiments, the administered composition reduces CAR-T cell toxicity in the subject. In some embodiments, the administered composition inhibits tumor growth or metastasis of a cancer in the subject.

In some embodiments, a pharmaceutical composition of the disclosure is administered to the subject individually (e.g., monotherapy) or as a first therapy in combination with a second therapy (multi-therapy). In some embodiments, the second therapy is selected from the group consisting of chemotherapy, radiotherapy, immunotherapy, hormonal therapy, toxin therapy, or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

In yet another aspect, provided herein are kits for modulating off-target T-cell activation in a subject, or treating a condition in a subject in need thereof, the kit including instructions for use thereof and one or more of the following: (a) one or more recombinant polypeptides of the disclosure; b) one or more nucleic acid constructs of the disclosure; c) one or more recombinant cell of the disclosure; and d) one or more pharmaceutical compositions of the disclosure.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1F schematically summarize the results of experiments performed to illustrate that anti-CD28 stimulation of CD19-CAR T cells is TMD dependent.

FIG. 1A is a schematic representation of five different designs of chimeric antigen receptors (CAR) against CD19 bearing a 4-1BB costimulatory domain and differing by their hinge and transmembrane domain.

FIG. 1B is a schematic representation of the experiments described in Example 2. FACS sorted CD4⁺CD127⁺ CD25^{low} T cells were electroporated with a CRISPR-Cas9 ribonucleoprotein complex (RNP) targeting the constant region of the TCRβ chain gene (TRBC), followed by stimulation with anti-CD3/CD28 beads (1:1 ratio).

FIG. 1C is a representative result of flow cytometry analysis of CD3 expression over time of cells electroporated with or without RNP. Percentages of residual CD3⁺ population and fold-expansion after 9 days of culture of CD4⁺ T cells electroporated with or without RNPs targeting TRBC are shown. Results shown in this figure are from four independent experiments.

FIG. 1D is a representative example of CFSE dilution of a mixed population of CD3^{+/-}CAR^{+/-} T re-stimulated with anti-CD3/28 beads.

FIG. 1E is a graph illustrating the expansion of CD3⁺ T cells that escaped TCR deletion. Normalized CFSE MFI ratio for CD3⁻mCherry⁺, CD3⁺mCherry⁻ and CD3⁺mCherry⁺ cells was calculated by dividing CFSE MFI of these populations with the MFI of the CD3⁻mCherry⁻ cells in the same culture. Two-way ANOVA was used for statistical analysis (bold line set as reference). Results shown are a summary of two independent experiments using T cells from 5 unrelated donors for each construct. ***p<0.001.

FIG. 1F illustrates the percentages of CD3⁺ and mCherry⁺ cells before and 5 days after re-stimulation of edited T cells with anti-CD3/CD28 beads. Unpaired t-test was performed comparing CD8-TMD and CD28-TMD containing CARs on D14. Results shown are a summary of 2 independent experiments using T cells from five unrelated donors for each construct. ***p<0.001.

FIGS. 2A-2C summarize the results of experiments performed to illustrate that CD28-TMD-containing CARs interact with CD28.

FIG. 2A illustrates a mixture of CFSE-labeled CD4⁺ T cells with or without CD3, CD28, and CAR expression. CFSE MFI of five independent donors in two independent experiments is reported. One-way ANOVA was used for statistical analysis.

FIG. 2B is a graph illustrating proliferation of purified CD3⁻CAR⁺ CD4⁺ T cells in response to plate-bound or soluble anti-CD28 stimulation. Results are representative of three independent experiments. Two-way ANOVA was used for statistical analysis.

FIG. 2C is Western blot analysis demonstrating interactions of CD28-TMD of the CAR with the endogenous CD28 receptor. CD28 or the Myc-tag of CD3⁻CAR⁺ T cells were immunoprecipitated. Western blot analysis of the input (5% of the whole cell lysate) as well as of the precipitated was performed using anti-CD28 (clone D2Z4E) and anti-Myc (clone 9B11). Results are representative of 2-3 independent experiments for each condition. **p<0.01***, p<0.001 Counts per minute (CPM). Not Statistically Significant (NS).

FIGS. 3A-3E summarize the results of experiments performed to illustrate that the dimerization of the CD28-TMD depends on a core of four amino acids.

FIG. 3A is a diagram representing the amino acid sequence of the wild type and four mutants of the CD28-TMD. A representative example of MYC and mCherry expression for each mutant is shown.

FIG. 3B illustrates representative examples of CFSE dilution of a mixed population of CD3^{+/-}CAR^{+/-}T cells re-stimulated with anti-CD3/CD28 beads.

FIG. 3C is a graph illustrating evaluation of various CD3⁻CAR⁺ cells with mutated CD28-TMD for their ability to proliferate in response to anti-CD28 stimulation. Normalized CFSE MFI ratio for CD3⁻CAR^{low/int/high} was calculated by dividing the MFI of each of these populations with the MFI of CD3⁻mCherry⁻ cells within the same culture. A summary of results using T cells from four unrelated donors in two independent experiments is shown.

FIG. 3D is a graph illustrating proliferation of purified CD3⁻CAR⁺ T cells in response to plate-bound anti-CD28 stimulation. Results represent the mean of three independent experiments.

FIG. 3E depicts a Western blot analysis of the input (5% of the whole cell lysate) as well as of the precipitate performed using anti-CD28 (clone D2Z4E) and anti-Myc (clone 9B11). CD28 or the Myc-tag of CD3⁻CAR⁺ T cells were immunoprecipitated. Results are representative of two independent experiments for each condition. Two-way ANOVA were used for statistical analysis. *p<0.05, **p<0.01***, p<0.001.

FIGS. 4A-4D schematically summarize the results of experiments demonstrating that CAR-CD28 heterodimers are B7-unresponsive but reduce CD28 expression.

FIG. 4A is a representative example showing CD71 upregulation in CAR T cells containing an IgG4-HD/CD28-TMD co-cultured for 48 hours with irradiated (4000Rad) CD19-wild type or deficient Raji cells with or without CTLA-4 Ig.

FIG. 4B illustrates the results of analyzing CD25⁺CD71⁺ T cells in low, intermediate (int), or high mCherry-expressing CAR-T cells using the gating strategy described in FIG. 9. Data were pooled from four independent experiments using T cells from 4-5 unrelated donors.

FIG. 4C is a schematic illustrating editing strategy and homology-directed repair-mediated integration into the TRAC locus of various CD19 CARs using an AAV-6 transduction protocol.

FIG. 4D illustrates Myc and CD28 expression in a representative example analyzed 6 days after editing and beads removal. A reduction in the CD28 mean fluorescence intensity was observed in both CD4+ and CD8+ T cells with CAR containing CD28ζ ICD.

FIG. 4E illustrates Myc and CD28 expression in a representative example analyzed 6 days after editing and beads removal. A reduction in the CD28 mean fluorescence intensity was observed in both CD4+ and CD8+ T cells with CAR containing 4-1BBζ ICD.

FIG. 4F illustrates CD28 MFI ratio calculated by dividing CD28 MFI of Myc+ cells by Myc− cells in the same culture. Pooled data from 3-4 independent experiments across five unrelated donors are shown. Each dot represents one independent editing condition. Two-way ANOVA was used for statistical analysis. *p<0.05, ***p<0.001.

FIG. 5 illustrates the sequence analysis of five CD19-CAR constructs differing by their hinge and transmembrane domains (see also, e.g., FIG. 1A). FMC63 on the far left represents the anti-CD19 single chain variable fragment (ScFv). 41BB on the far right represents the 4-1BB intracellular domains. The bars between FMC63 and 41BB represents transmembrane predictions. Transmembrane probability (height of grey bars) was determined using a web-based tool for topology prediction of transmembrane helices in polypeptide sequences based on a hidden Markov model (www.cbs.dtu.dk/services/TMHMM/).

FIGS. 6A-6C summarize the results of experiments performed to illustrate anti-CD19 CAR expression on CD4 T cells.

FIG. 6A is flow cytometry analysis (top row) of the 5 different CAR constructs showing the transduction profile of Cherry⁺ and Myc⁺ among CD4⁺ T cells. The bottom row illustrates flow dot plot overlays of T-cell activation (measured by CD25 and CD71 expression) when cultured with (gray) or without (red) CD19-expressing K562 (K562-CD19) cells.

FIG. 6B illustrates a summary of the percentage of mCherry⁺ cells and mCherry MFI (gated on mCherry positive cells) from three independent experiments using T cells from six independent donors is shown. One-way ANOVA was used for statistical analysis.

FIG. 6C is a graph illustrating CD25+ CD71+ expression among CD4⁺ T cells analyzed by comparing T cells cultured alone or with CD19-expressing K562 cells. A summary of the results from two independent experiments using T cells from three independent donors is graphed. Two-way ANOVA analysis was used for statistical analysis, *p<0.05, **p<0.01, ***p<0.001.

FIGS. 7A-7D schematically summarize the results of experiments performed to investigate the proliferation of CD19-CAR T cells expressing a CD28ζ or 4-1BBζ intracellular domain.

FIG. 7A schematically describes the design of CARs constructs bearing an IgG4 hinge, CD28 transmembrane (TMD) domain with either a CD28 CD3ζ or 4-1BB CD3ζ intracellular domain (ICD). Both CARs are fused to a T2A-EGFRt reporter.

FIG. 7B is representative plots illustrating editing efficiency 6 days after transduction.

FIG. 7C is a graph illustrating normalized CFSE MFI ratio. On day 9 of culture, a mixed population of CD3^{+/-}CAR^{+/-} cells was labeled with CFSE and re-stimulated with anti-CD3/CD28 beads and cultured for 4 days. Normalized CFSE MFI for CD3⁺EGFRt⁻, CD3⁺EGFRt⁺ and CD3⁻ CAR⁺ cells were calculated by dividing CFSE MFI of these populations with the MFI of the CD3⁻CAR⁻ cells in the same culture. Results from 4-6 independent donors from three independent experiments are summarized. One-way ANOVA was used for statistical analysis.

FIG. 7D illustrates percentage comparisons of CD3 and CAR expression before and after re-stimulation. On day 9 of culture, a mixed population of CD3^{+/-}CAR^{+/-} cells was re-stimulated with anti-CD3/CD28 beads and IL-2 (30 IU/mL) and expanded for another 5 days. The percentage of CD3 and CAR expression was compared before (D9) and after re-stimulation (D14) by flow cytometry. Results from five independent donors from three independent experiments are summarized. **p<0.01, ***p<0.001.

FIGS. 8A-8B schematically summarize the results of experiments performed to investigate the anti-CD28 dependent proliferation and cytokine production of CD19-CAR T cells expressing a CD28ζ or 4-1BBζ intracellular domain.

FIG. 8A is a graph illustrating proliferation assessment. CD3⁻CD4⁺ T cells expressing CD19-CAR engineered with an IgG4-HD/CD28-TMD-28ζ-ICD or IgG4-HD/28-TMD-4-1BBζ-ICD CAR were FACS purified based on EGFRt expression. Cells were stimulated with soluble anti-CD28 (clone CD28.2, 1 μg/mL), soluble anti-CD3 (clone HIT3α, 2 μg/mL), or no stimulation. Proliferation was assessed using 3H-thymidine incorporation 64 hours later, with 3H thymidine added during the last 16-18 hours. One-way ANOVA was used for statistical analysis. Representative results of 4-7 independent experiments for each condition are shown. One-way ANOVA was used for statistical analysis.

FIG. 8B illustrates cytokines secretion during the first 48 hours after stimulation with soluble anti-CD28 (clone CD28.2, 1 μg/mL) measured using multiplexed Luminex. Results units are pg/mL and are a summary of 4-7 independent experiments using T cells from four independent donors. ***p<0.001.

FIGS. 9A-9B schematically depict the results of experiments illustrating mixed lymphocyte reaction with CD19-deficient Raji cells.

FIG. 9A illustrates representative staining for CD80, CD86, HLA-DR4 and CD19 of Raji cells. CAR T cells were stimulated with different HD and TMD with CD19-deficient Raji cells that express high levels of CD80 and CD86.

FIG. 9B illustrates that CD19-deficient Raji induced CAR T activation. CAR T cells were co-cultured alone, with wild type or with CD19-deficient irradiated Raji (4000 rad) cells at 1:1 ratio with or without CTLA-4 Ig. Gating strategy used to define the percentage of CD25⁺CD71⁺ expression among the mCherry low (l), intermediate (i), and high (h) CD4⁺CD3⁻ cells.

FIGS. 10A-10B schematically summarize the results of experiments performed illustrating AAV-transduced CAR expression and proliferation.

FIG. 10A illustrates that the level of the CAR expression was similar regardless of the differences in editing efficiency. Transduction efficiency was defined by the percentage of MYC⁺CD3⁻ cells. Following electroporation, AAV6 viruses were tittered resulting in different transduction efficiencies. MYC MFI was defined for each condition. The titration for the CAR constructs bearing a CD8-HD or an IgG4-HD were performed on two separate experiments with two independent donors.

FIG. 10B illustrates that all CAR T cells proliferated upon stimulation with CD19+ NALM-6 target cells. On Day 8 of culture, edited and unedited T cells were stained for CFSE and co-cultured with NALM-6 cells for 4 days. The gating strategy and CFSE dilutions are shown. A representative example of two independent experiments is shown.

FIG. 11 is a schematic illustration of CARs containing a CD28-TMD in accordance with some embodiments of the disclosure form heterodimers with the endogenous CD28 in human T cells. This dimerization was found to be dependent on polar amino acids in the CD28-TMD. The CD28-CAR heterodimers did not respond to CD80 and CD86 stimulation.

FIGS. 12A-12F summarizes the results of hinge-hinge interaction modeling.

FIG. 12A illustrates modeling of a CD28-CAR heterodimer. The extracellular part of the CD28 receptor and CAR engineered with a CD28 HD is shown. The start of the transmembrane domain is represented by gray circles.

FIG. 12B illustrates modeling of a CD28-CAR heterodimer. The extracellular part of the CD28 receptor and CAR engineered with an IgG4 HD is shown. The start of the transmembrane domain is represented by gray circles.

FIG. 12C illustrates modeling of a CD28-CAR heterodimer. The extracellular part of the CD28 receptor and CAR engineered with a CD8 HD is shown. The start of the transmembrane domain is represented by gray circles.

FIG. 12D illustrates modeling of a CAR-CAR homodimer. CAR engineered with CD28 HD is shown. The start of the transmembrane domain is represented by gray circles.

FIG. 12E illustrates modeling of a CAR-CAR homodimer. CAR engineered with IgG4 HD is shown. The start of the transmembrane domain is represented by gray circles.

FIG. 12F illustrates modeling of a CAR-CAR homodimer. CAR engineered with CD8 HD is shown. The start of the transmembrane domain is represented by gray circles.

FIGS. 13A-13B show a representative example of two independent experiments of CFSE dilution of CD3−CAR+ T cells re-stimulated with anti-CD3/28 beads (FIG. 13A) or left unstimulated (FIG. 13B).

### DETAILED DESCRIPTION OF THE DISCLOSURE

The present disclosure relates generally to chimeric polypeptides, including chimeric antigen receptors (CARs), which include a transmembrane domain from CD28 and a hinge domain. In some embodiments, the hinge domain is capable of promoting dimerization of the chimeric polypeptides. The disclosure also provides compositions and methods useful for making such polypeptides and CARs, as well as methods for the detection and treatment of relevant health conditions, such as proliferative diseases (e.g., cancer).

As described above, CAR-engineered T cells are emerging as promising therapies for otherwise untreatable diseases. It is worth noting that in earlier iterations of CAR products, e.g., tisagenlecleucel, axicabtagene ciloleucel, and Lisocabtagene Maraleucel, differ in their hinge domain (HD) and transmembrane domain (TMD), CD28-HD/TMD for KTE-19, CD8-HD/TMD for CTL-019, and IgG4-HD/CD28-TMD for JCAR-17.

The mechanisms underlying the differences between CD8-HD/TMD and CD28-HD/TMD domains however remain to be defined. For example, although anti-CD19 chimeric antigen receptor (CD19-CAR)-engineered T cells are approved therapeutics for malignancies, the impact of the hinge (HD) and transmembrane (TMD) domains between the extracellular antigen-targeting and the intracellular signaling modalities of CARs have not been systemically studied. As described in greater detail below, an investigation of the impact of CD28-TMD on CD19-CARs has made the surprising discovery that CD28-TMD mediated a heterodimeric association of the CAR with the endogenous CD28 receptor. In particular, a series of CD19-CARs differing only by their HD (CD8/CD28/IgG4) and TMD (CD8/CD28) was generated. CARs containing a CD28-TMD, but not a CD8-TMD, formed heterodimers with the endogenous CD28, as shown by co-immunoprecipitation and CAR-dependent proliferation to anti-CD28 stimulation alone. Dimerization depended on a core of four amino acids in the CD28-TMD. CAR-CD28 heterodimerization was more efficient in CARs containing a CD8-HD or CD28-HD as compared to an IgG4-HD. CD28-CAR heterodimers did not respond to CD80 and CD86 stimulation yet could drive a 26-51% reduction in CD28 cell-surface expression. These data unveil a new property of the CD28-TMD and indicate that TMDs can modulate CAR T-cell activities by engaging endogenous partners, which can lead to promotion of survival and homeostasis of CAR-T cells, particularly in the absence of CAR targets.

Furthermore, as described in greater detail below, an investigation of the influence of the hinge domain on the interaction of CD28 with CARs indicates that the cysteine bridge in the CD28-HD is insufficient to mediate CD28-CAR heterodimerization without dimerization in the CD28-TMD (see, e.g., Example 5). These results demonstrate that cysteines and inter-molecular disulfide bonds in the HDs are not the drivers of CAR-CD28 heterodimerization but can also be involved in the stabilization of the CAR-CD28 heterodimers.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The terms “administration” and “administering”, as used herein, refer to the delivery of a bioactive composition or formulation by an administration route comprising, but not limited to, intranasal, transdermal, intravenous, intra-arterial, intramuscular, intraperitoneal, subcutaneous, intramuscular, oral, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “effective amount”, “therapeutically effective amount”, or “pharmaceutically effective amount” of a composition of the disclosure, e.g., DIP, nucleic acid construct, or pharmaceutical composition, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions” to each other.

Within the meaning of the term “equivalent amino acid substitution” as applied herein, one amino acid may be substituted for another without substantially altering the structure and/or functionality of the polypeptide. Exemplary equivalent or conserved amino acid substitutions are within the groups of amino acids indicated herein below:
i) Amino acids having polar side chains (Asp, Glu, Lys, Arg, His, Asn, Gln, Ser, Thr, Tyr, and Cys);
ii) Amino acids having non-polar side chains (Gly, Ala, Val, Leu, Ile, Phe, Trp, Pro, and Met);
iii) Amino acids having aliphatic side chains (Gly, Ala Val, Leu, Ile);
iv) Amino acids having cyclic side chains (Phe, Tyr, Trp, His, Pro);
v) Amino acids having aromatic side chains (Phe, Tyr, Trp);
vi) Amino acids having acidic side chains (Asp, Glu);
vii) Amino acids having basic side chains (Lys, Arg, His);
viii) Amino acids having amide side chains (Asn, Gln);
ix) Amino acids having hydroxy side chains (Ser, Thr);
x) Amino acids having sulphur-containing side chains (Cys, Met);
xi) Neutral, weakly hydrophobic amino acids (Pro, Ala, Gly, Ser, Thr);
xii) Hydrophilic, acidic amino acids (Gin, Asn, Glu, Asp); and
xiii) Hydrophobic amino acids (Leu, Ile, Val).

In some embodiments, a Point Accepted Mutation (PAM) matrix is used to determine equivalent amino acid substitutions. In some embodiments, a BLOck SUbstitution Matrix (BLOSUM) is used to determine equivalent amino acid substitutions.

As used herein, the term “functional fragment thereof” or “functional variant thereof” refers to a molecule having quantitative and/or qualitative biological activity in common with the wild-type molecule from which the fragment or variant was derived. For example, a functional fragment or a functional variant of a hinge domain is one which retains essentially the same ability to promote oligomerization, e.g., dimerization of the chimeric polypeptides and CARs as described herein, as compared to the hinge domain from which the functional fragment or functional variant was derived. In some embodiments, a functional fragment or a functional variant of a hinge domain is one which retains essentially the same ability to promote oligomerization, e.g., dimerization of the chimeric polypeptides and CARs as described herein via intermolecular disulfide bonding, as compared to the hinge domain from which the functional fragment or functional variant was derived. In another example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about” which, as used herein, has its ordinary meaning of approximately. The term “about” is used to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

The term “construct” refers to a recombinant molecule including one or more isolated nucleic acid sequences from heterologous sources. For example, nucleic acid constructs can be chimeric nucleic acid molecules in which two or more nucleic acid sequences of different origin are assembled into a single nucleic acid molecule. Thus, representative nucleic acid constructs include any constructs that contain (1) nucleic acid sequences, including regulatory and coding sequences that are not found adjoined to one another in nature (e.g., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative nucleic acid constructs can include any recombinant nucleic acid molecules, linear or circular, single stranded or double stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Constructs of the present disclosure can include the necessary elements to direct expression of a nucleic acid sequence of interest that is also contained in the construct. Such elements may include control elements such as a promoter that is operably linked to (so as to direct transcription of) the nucleic acid sequence of interest, and optionally includes a polyadenylation sequence. In some embodiments of the disclosure, the nucleic acid construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in a cell. For the practice of the present disclosure, compositions and methods for preparing and using constructs and cells are known to one skilled in the art.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that, operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid constructs disclosed herein may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions of the Disclosure

As described in greater detail herein, one aspect of the present disclosure relates to chimeric polypeptides and chimeric antigen receptors (CARs) that include a CD28 transmembrane domain (CD28-TMD), such as a human CD28-TMD. In some embodiments, the chimeric polypeptides and CARs of the disclosure further include a hinge domain inserted between the transmembrane domain and an extracellular antigen-binding moiety of the chimeric polypeptides and CARs. In some embodiments, the hinge domain is capable of promoting dimerization of the chimeric polypeptides and CARs. Also provided are nucleic acid constructs encoding such chimeric polypeptides, as well as recombinant cells that have been engineered to express a chimeric polypeptide or CAR as disclosed herein and are directed against a cell of interest such as a cancer cell.

### Chimeric Polypeptides

In one aspect, some embodiments disclosed herein relate to chimeric polypeptides which contain a transmembrane domain (TMD) derived from CD28. In some embodiments, the chimeric polypeptides of the disclosure contain a CD28-TMD. In some embodiments, the chimeric polypeptides of the disclosure further include a hinge domain capable of promoting dimerization of the chimeric polypeptides. In some embodiments, the chimeric polypeptides of the disclosure include: (a) an extracellular domain (ECD) having a binding affinity for an antigen; (b) a hinge domain capable of promoting dimerization of the chimeric polypeptide; (c) a CD28-TMD; and (d) an intracellular signaling domain (ICD).

### Extracellular Domain (ECD)

In some embodiments, the ECD of the chimeric polypeptides disclosed herein has a binding affinity for one or more target antigens. In some embodiments, the ECD includes an antigen-binding moiety capable of binding to one or more antigens on the surface of a cell. In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment of any thereof. In some embodiments, the antigen-binding moiety of the ECD includes a scFv.

The antigen-binding moiety of the ECD can include naturally-occurring amino acid sequences or can be engineered, designed, or modified so as to provide desired and/or improved properties, e.g., binding affinity. Generally, the binding affinity of an ECD, e.g., antibody or an antigen-binding moiety of an ECD for a target antigen (e.g., CD19 antigen) can be calculated by the Scatchard method described by Frankel et al., Mol. Immunol, 16: 101-106, 1979. In some embodiments, binding affinity can be measured by an antigen/antibody dissociation rate. In some embodiments, a high binding affinity can be measured by a competition radioimmunoassay. In some embodiments, binding affinity can be measured by ELISA. In some embodiments, the binding affinity of an ECD, e.g., antibody or an antigen-binding moiety of an ECD for a target antigen (e.g., CD19 antigen) can be measured by real-time, label free bio-layer interferometry assay, for example, at 25° C. or 37° C., e.g., an Octet® HTX biosensor, or by surface plasmon resonance (SPR), e.g., BIACORE™, or by solution-affinity ELISA. In some embodiments, binding affinity can be measured by flow cytometry. An antigen-binding moiety that “selectively binds” a target antigen (such as CD19) is a moiety that binds the target antigen with high affinity and does not significantly bind other unrelated antigens but binds the antigen with high affinity, e.g., with an equilibrium constant (KD) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

The binding of the antigen-binding moiety to its target can be either in a competitive or non-competitive fashion with a natural ligand of the target antigen. Accordingly, in some embodiments of the disclosure, the binding of the antigen-binding moiety to its target antigen can be ligand-blocking. In some other embodiments, the binding of the antigen-binding moiety to its target antigen does not block binding of the natural ligand.

A skilled artisan can select an ECD based on the desired localization or function of a cell that is genetically modified to express a chimeric polypeptide of the present disclosure. For example, a chimeric polypeptide with an ECD including an antibody specific for a CD19 antigen can target recombinant CAR-T cells to CD19-expressing B cells, and can target cancers that arise from this type of cells, such as B cell lymphomas, acute lymphoblastic leukemia (ALL), and chronic lymphocytic leukemia (CLL). In some embodiments, the ECD of the chimeric polypeptides disclosed herein is capable of binding a tumor-associated antigen (TAA) or a tumor-specific antigen (TSA). A skilled artisan will understand that a TAA is generally a molecule, such as e.g., protein, present on tumor cells and on normal cells, or on many normal cells, but at much lower concentration than on tumor cells. In contrast, a TSA is generally a molecule, such as e.g., protein which is present on tumor cells but absent from normal cells.

### Antigens

In principle, there are no particular limitations with regard to suitable target antigens. In some embodiments of the disclosure, the antigen-binding moiety of the ECD is specific for an epitope present in an antigen that is expressed by a tumor cell, i.e., a tumor-associated antigen. The tumor-associated antigen can be an antigen associated with, e.g., a pancreatic cancer cell, a colon cancer cell, an ovarian cancer cell, a prostate cancer cell, a lung cancer cell, mesothelioma cell, a breast cancer cell, a urothelial cancer cell, a liver cancer cell, a head and neck cancer cell, a sarcoma cell, a cervical cancer cell, a stomach cancer cell, a gastric cancer cell, a melanoma cell, a uveal melanoma cell, a cholangiocarcinoma cell, a multiple myeloma cell, a leukemia cell, a lymphoma cell, and a glioblastoma cell. In some embodiments, the antigen-binding moiety is specific for an epitope present in a tissue-specific antigen. In some embodiments, the antigen-binding moiety is specific for an epitope present in a disease-associated antigen.

Examples of antigens suitable for the compositions and methods disclosed herein include autoantigens and neoantigens present at site of inflammation, as well as transplant antigens in the context of regulatory T (Treg) cell therapy. Several autoantigens are suitable, and generally include those selectively expressed in tissue affected by autoimmune diseases, such as myelin basic protein in the brain for autoimmune and inflammatory diseases in the brain including MS, ALS, desmogleins (e.g., DSG1, DSG2, DSG3, and DSG4) for skin diseases. Non-limiting examples of neoantigens suitable for the compositions and methods disclosed herein include new antigens produced by inflammation or exposed by tissue damage. Non-limiting examples of transplant antigens include HLAs that are mismatched between the donor and the recipients, which can be MHC class I such as HLA-A2 or HLA class II, DR, DO, and DQ.

Non-limiting examples of suitable target antigens include CD19, HLA-A2 (A2), Glypican 2 (GPC2), human epidermal growth factor receptor 2 (Her2/neu), CD276 (B7-H3), IL-13-receptor alpha 1, IL-13-receptor alpha 2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional antigens that can be suitable for the chimeric polypeptides and CARs disclosed herein include, but are not limited to, the dimeric form of the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is CD19. In some embodiments, the antigen is HLA-A2.

### Hinge Domain

As described above, the chimeric polypeptides and CARs of the disclosure include a hinge domain inserted between the transmembrane domain and an extracellular antigen-binding moiety of the chimeric polypeptides and CARs. Without being bound to any particular theory, the hinge domain of the chimeric polypeptides and CARs disclosed herein serves several functions, including controlling flexibility and rigidity of the chimeric polypeptides and CARs, which in turn can affect antigen binding and signal transduction. The length of the hinge domain can be tuned to enhance the CARs' ability to reach antigens in the space between a CAR T cell and a target cell. The hinge domain can also be tuned to mediate dimerization. Care should be taken in selecting a suitable hinge domain. For example, shorter hinge domain, e.g., IgG4 is more rigid and more effective at transducing signal once engaged on target antigen but may not reach target that are more membrane proximal or otherwise specially constrained. In some embodiments, the hinge domain is capable of promoting oligomerization, e.g., dimerization of the chimeric polypeptides and CARs. In some embodiments, the hinge domain promotes oligomer, e.g., dimer formation of the chimeric polypeptides and CARs via intermolecular disulfide bonding. In these instances, within the chimeric polypeptides and CARs disclosed herein, the hinge domain generally includes a flexible polypeptide connector region disposed between the ECD and the TMD. In some embodiments, the hinge domain includes motifs that promote dimer formation of the chimeric polypeptides disclosed herein. Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant of any thereof. In some embodiments, the hinge polypeptide sequence contains one or more CXXC motifs. In some embodiments, the hinge polypeptide sequence contains one or more CPPC motifs. Additional information in this regard can be found in, for example, a recent review by G. Vidarsson et al., Frontiers Immunol (2014) 5:520 (doi: 10.3389/fimmu.2014.00520), which is hereby incorporated by reference in its entirety.

Hinge polypeptide sequences can also be derived from a CD8α hinge domain, a CD28 hinge domain, an IgG4 hinge domain, and an Ig4 CH2-CH3 domain, and functional variants thereof. In some embodiments, the hinge domain includes a hinge polypeptide sequence derived from a CD8α hinge domain or a functional variant thereof. In some embodiments, the hinge domain includes a hinge polypeptide sequence derived from a CD8α hinge domain and includes an amino acid sequence having at least 80%, at least 85%, at least 90%, at least 95%, at least 96%, at least 97%, at least 98%, at least 99%, or 100% sequence identify to the amino acid sequence of SEQ ID NO: 12. In some embodiments, the hinge domain includes the amino acid sequence of SEQ ID NO: 12, wherein one, two, three, four, or five amino acid residues in the sequence of SEQ ID NO: 12 is optionally substituted by a different amino acid residue. In some embodiments, the hinge domain includes a hinge polypeptide sequence derived from a CD28 hinge domain or a functional variant thereof. In some embodiments, the hinge domain includes a hinge polypeptide sequence derived from a CD28 hinge domain and includes an amino acid sequence having at least 80%, at least 85%, at least 90%, at least 95%, at least 96%, at least 97%, at least 98%, at least 99%, or 100% sequence identify to the amino acid sequence of SEQ ID NO: 13. In some embodiments, the hinge domain includes a hinge polypeptide sequence derived from an IgG4 hinge domain or a functional variant thereof. In some embodiments, the hinge domain includes a hinge polypeptide sequence derived from a CD28 hinge domain and includes an amino acid sequence having at least 80%, at least 85%, at least 90%, at least 95%, at least 96%, at least 97%, at least 98%, at least 99%, or 100% sequence identify to the amino acid sequence of SEQ ID NO: 11. In some embodiments, the hinge domain includes a hinge polypeptide sequence derived from an Ig4 CH2-CH3 domain or a functional variant thereof.

### Transmembrane Domain (TMD)

As described above, the chimeric polypeptides and CARs of the disclosure include a transmembrane domain derived from CD28. One skilled in the art will understand that the term “derived from” when used in reference to a polypeptide molecule, such as CD28-TMD polypeptide molecule, refers to the origin or source of the polypeptide molecule, and may include naturally occurring, recombinant, unpurified, or purified molecules. Polypeptide molecules are considered “derived from” when they include portions or elements assembled in such a way that they produce a functional polypeptide. The portions or elements can be assembled from multiple sources provided that they retain evolutionarily conserved function. In some embodiments, the derivative CD28-TMD polypeptide molecules of the disclosure include substantially the same sequence as the source CD28-TMD polypeptide molecule. For example, the derivative CD28-TMDs of the present disclosure may have at least 80%, 85%, 90%, 95%, 98%, 99%, or 100% sequence identity to the source CD28-TMD polypeptide and still retain evolutionarily conserved function of a CD28-TMD.

In some embodiments, the chimeric polypeptides and CARs of the disclosure include a CD28-TMD. In some embodiments, the CD28-TMD is a human CD28 TMD. In some embodiments, the CD28-TMD is from different mammalian species, e.g., non-human mammals such as, mouse CD28 or non-human primate CD28. In some embodiments, the TMD includes one or more amino acid substitutions within a transmembrane dimerization motif of the CD28-TMD. In some embodiments, the transmembrane dimerization motif of the CD28-TMD comprises the consensus sequence YxxxT. In some embodiments, the TMD includes an amino acid sequence having at least 70%, at least 80%, at least 85%, at least 90%, at least 95%, at least 96%, at least 97%, at least 98%, at least 99%, or 100% sequence identity to a CD28-TMD having the sequence of SEQ ID NO: 1, and further includes one or more amino acid substitutions at a position corresponding to an amino acid residue selected from the group consisting of X13, X14, X15, and X19 of SEQ ID NO: 1. In some embodiments, the TMD includes the sequence of SEQ ID NO: 1, and further includes one or more amino acid substitutions at an amino acid residue selected from the group consisting of C13, Y14, S15, and T19 of SEQ ID NO: 1. In some embodiments, the TMD includes the sequence of SEQ ID NO: 1, and further includes one, two, three, four, or five amino acid residues in the sequence of SEQ ID NO: 1 is optionally substituted by a different amino acid residue. In some embodiments, one, two, three, four, or five amino acid residues in the sequence of SEQ ID NO: 1 is optionally substituted by an equivalent amino acid residue. In some embodiments, the TMD includes the sequence of SEQ ID NO: 1.

In some embodiments, the TMD includes an amino acid sequence having at least 70%, at least 80%, at least 85%, at least 90%, at least 95%, at least 96%, at least 97%, at least 98%, at least 99%, or 100% sequence identity to a CD28-TMD having the sequence of SEQ ID NO: 2, and further includes one or more amino acid substitutions at a position corresponding to an amino acid residue selected from the group consisting of X13, X14, X15, and X19 of SEQ ID NO: 2. In some embodiments, the TMD includes the sequence of SEQ ID NO: 2, and further includes one, two, three, four, or five amino acid residues in the sequence of SEQ ID NO: 2 is optionally substituted by a different amino acid residue. In some embodiments, one, two, three, four, or five amino acid residues in the sequence of SEQ ID NO: 2 is optionally substituted by an equivalent amino acid residue. In some embodiments, the TMD includes the sequence of SEQ ID NO: 2. In some embodiments, the TMD includes the sequence of SEQ ID NO: 3. In some embodiments, the TMD includes the sequence of SEQ ID NO: 4. In some embodiments, the TMD includes the sequence of SEQ ID NO: 5. In some embodiments, the TMD includes the sequence of SEQ ID NO: 6.

In some embodiments, the one or more amino acid substitutions is independently selected from the group consisting of a leucine substitution, an alanine substitution, an arginine substitution, an aspartic acid substitution, a histidine substitution, a glutamic acid substitution, a lysine substitution, a serine substitution, a tryptophan substitution, and combinations of any thereof. In some embodiments, at least one of the one or more amino acid substitutions is a nonpolar-to-polar amino acid substitution. In some embodiments, at least one of the one or more amino acid substitutions is a nonpolar-to-polar amino acid substitution. In some embodiments, the one or more amino acid substitutions results in reduced binding of the chimeric polypeptide to a CD28 polypeptide compared to binding of a chimeric polypeptide that includes a CD28-TMD lacking the one or more amino acid substitution. In some embodiments, the amino acid substitution at position X13 is a Cys-to-Leu substitution (C13L). In some embodiments, the amino acid substitution at position X14 is a Tyr-to-Leu substitution (Y14L). In some embodiments, the amino acid substitution at position X15 is a Ser-to-Leu substitution (S15L). In some embodiments, the amino acid substitution at position X15 is a Thr-to-Leu substitution (T19L). In some embodiments, the TMD includes the sequence of SEQ ID NO: 1, and further includes the following amino acid substitutions: C13L, Y14L, S15L, and T19L.

### Intracellular Domain (ICD)

As described above, the ICD of the chimeric polypeptides and CARs disclosed herein includes one or more costimulatory domains. Generally, the costimulatory domain suitable for the chimeric polypeptides and CARs disclosed herein can be any one of the costimulatory domains and functional variants thereof known in the art. Examples of suitable costimulatory domains that can enhance cytokine production and include, but are not limited to, costimulatory polypeptide sequences derived from 4-1BB (CD137), CD27 (TNFRSF7), CD28, CD70, LFA-2 (CD2), CD5, ICAM-1 (CD54), ICOS, LFA-1 (CD11a/CD18), DAP10, and DAP12. In some embodiments, the ICD of the chimeric polypeptides and CARs disclosed herein includes a costimulatory sequence derived from 4-1BB. In some embodiments, the costimulatory sequence is derived from a 4-1BB protein and includes an amino acid sequence having at least 70%, at least 80%, at least 85%, at least 90%, at least 95%, at least 96%, at least 97%, at least 98%, at least 99%, or 100% sequence identity to the sequence of SEQ ID NO: 15. In some embodiments, the ICD includes two costimulatory domains.

In some embodiments of the disclosure, the ICD of the disclosed chimeric polypeptides and CARs includes conserved amino acid motifs that serve as substrates for phosphorylation such as, for example, immunoreceptor tyrosine-based activation motifs (ITAM), and/or immunoreceptor tyrosine-based inhibition motifs (ITIM). In some embodiments, the ICD of the disclosed chimeric polypeptides and CARs includes at least 1, at least 2, at least 3, at least 4, or at least 5 specific tyrosine-based motifs selected from ITAM motifs, an ITIM motifs, or related intracellular motifs that serve as a substrate for phosphorylation. In some embodiments of the disclosure, the ICD of the disclosed chimeric polypeptides and CARs includes at least 1, at least 2, at least 3, at least 4, or at least 5 ITAMs. Generally, any ICD including an ITAM can be suitably used for the construction of the chimeric polypeptides as described herein. An ITAM generally includes a conserved protein motif that is often present in the tail portion of signaling molecules expressed in many immune cells. The motif may include two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some embodiments, the ICD comprising at least 1, at least 2, at least 3, at least 4, or at least 5 ITAMs independently selected from the ITAMs derived from CD3ζ, FcRγ, and combinations thereof. In some embodiments, the ICDs of the disclosed chimeric polypeptides and CARs comprises a CD3ζ ICD or a functional variant thereof. In some embodiments, the CD3ζ ICD includes an amino acid sequence having at least 70%, at least 80%, at least 85%, at least 90%, at least 95%, at least 96%, at least 97%, at least 98%, at least 99%, or 100% sequence identity to the sequence of SEQ ID NO: 17. In some embodiments, the CD3ζ ICD includes the amino acid sequence of SEQ ID NO: 17, wherein one, two, three, four, or five amino acid residues in the sequence of SEQ ID NO: 17 is optionally substituted by a different amino acid residue. In some embodiments, the CD3ζ ICD includes an amino acid sequence having at least 70%, at least 80%, at least 85%, at least 90%, at least 95%, at least 96%, at least 97%, at least 98%, at least 99%, or 100% sequence identity to the sequence of SEQ ID NO: 18. In some embodiments, the CD3ζ ICD includes the amino acid sequence of SEQ ID NO: 18, wherein one, two, three, four, or five amino acid residues in the sequence of SEQ ID NO: 18 is optionally substituted by a different amino acid residue. In some embodiments, one or more of the amino acid substitutions in the ICD is an equivalent amino acid substitution. In some embodiments, one or more of the amino acid substitutions in the ICD is independently selected from the group consisting of a leucine substitution, an alanine substitution, an arginine substitution, an aspartic acid substitution, a histidine substitution, a glutamic acid substitution, a lysine substitution, a serine substitution, a tryptophan substitution, and combinations of any thereof.

In some embodiments, the chimeric polypeptide of the disclosure includes at least one polypeptide domain operably linked to a second polypeptide domain to which it is not naturally linked in nature. The chimeric polypeptide domains may normally exist in separate proteins that are brought together in the chimeric polypeptide disclosed herein or they may normally exist in the same protein but are placed in a new arrangement in the chimeric polypeptide disclosed herein. A chimeric polypeptide as disclosed herein may be created, for example, by chemical synthesis, or by creating and translating a chimeric polynucleotide in which the polypeptide domains are encoded in the desired relationship.

In some embodiments, at least two of the polypeptide domains are directly linked to one another. In some embodiments, all of the polypeptide domains are directly linked to one another. In some embodiments, at least two of the polypeptide domains are directly linked to one another via at least one covalent bond. In some embodiments, at least two of the polypeptide domains are directly linked to one another via at least one peptide bond. In some embodiments, the chimeric polypeptides of the disclosure include one or more linkers which join the two or more polypeptide domains together. In some embodiments, at least two of the polypeptide domains are operably linked to one another via a linker. There is no particular limitation on the linkers that can be used in the chimeric polypeptides described herein. In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The linker can also be a linker peptide sequence. Accordingly, in some embodiments, at least two of the polypeptide domains are operably linked to one another via a linker peptide sequence. In principle, there are no particular limitations to the length and/or amino acid composition of the linker peptide sequence. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues.

### Nucleic Acid Constructs

In outlined above, one aspect of the disclosure relates to nucleic acid constructs including a nucleic acid sequence encoding a chimeric polypeptide or CAR of the disclosure, including expression cassettes, and expression vectors containing these nucleic acid constructs operably linked to heterologous nucleic acid sequences such as, for example, regulator sequences which allow in vivo expression of the chimeric polypeptide in a host cell or ex-vivo cell-free expression system.

Nucleic acid molecules of the present disclosure can be nucleic acid molecules of any length, including nucleic acid molecules that are generally between about 200 bp to about 2000 bp, e.g., between about 200 bp to about 1000 bp, between about 300 bp to about 1200 bp, between about 400 bp to about 1400 bp, between about 500 bp to about 1600 bp, between about 600 bp to about 1800 bp, between about 700 bp to about 2000 bp, between about 200 bp to about 500 bp, or between about 400 bp to about 1200 bp, for example between about 400 bp to 800 bp, between about 500 bp to about 1000 bp, between about 600 bp to about 800 bp, about 700 bp to about 1100 bp, or about 800 bp to about 1200 bp. In some embodiments, the nucleic acid molecules of the present disclosure can be between about 0.5 Kb and about 50 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb. In some embodiments, the nucleic acid molecules of the disclosure are between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb. The basic techniques for operably linking two or more sequences of DNA together are familiar to the skilled worker, and such methods have been described in a number of texts for standard molecular biological manipulation. The molecular techniques and methods by which these new nucleic acid molecules were constructed and characterized are described more fully in the Examples herein.

In some embodiments, the recombinant nucleic acid construct is operably linked to a heterologous nucleic acid sequence. In some embodiments, the heterologous nucleic acid sequence is a promoter.

In some embodiments, the recombinant nucleic acid construct is further defined as an expression cassette or a vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric polypeptide as disclosed herein, which is operably linked to expression control elements, such as a promoter, and optionally, any other sequences or a combination of other nucleic acid sequences that affect the transcription or translation of the coding sequence.

In some embodiments, the nucleic acid construct is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the vector is a vector derived from a lentivirus, an adeno virus, an adeno-associated virus, a baculovirus, or a retrovirus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In addition or alternatively, two or more separate chimeric polypeptides or CARs can be expressed on a single T cell using a single vector by taking advantage of ribosomal skip sequences or internal ribosomal entry sites (“bicistronic CAR”). Accordingly, in some embodiments, nucleic acid constructs of the disclosure can encode two or more chimeric polypeptides or CARs as disclosed herein. For example, a nucleic acid that encodes two or more chimeric polypeptides or CARs can be a multi-cistronic nucleic acid, wherein the two or more coding sequences are separated by a sequence encoding an IRES (internal ribosome entry site), which provide for expression of each chimeric polypeptide or CAR separately, or for the immediate cleavage into two separate chimeric polypeptides upon expression.

In some embodiments, a nucleic acid construct of the disclosure further includes a coding sequence for an autoproteolytic peptide. In some embodiments, the autoproteolytic peptide includes one or more autoproteolytic cleavage sites derived from calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof. In some embodiments, the coding sequence for an autoproteolytic peptide is operably linked downstream to the costimulatory domain or downstream to the CD3ζ ICD. In some embodiments, the coding sequence for an autoproteolytic peptide is operably linked upstream to a reporter gene (e.g., mCherry). In some embodiments, the coding sequence for an autoproteolytic peptide is derived from a porcine teschovirus-1 2A (P2A). In some embodiments, the coding sequence for an autoproteolytic peptide is derived from a Thosea asigna virus 2A (T2A).

The nucleic acid sequences encoding the chimeric polypeptides and CARs of the disclosure can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

In some embodiments, the recombinant nucleic acid of the disclosure includes a nucleic acid sequence encoding a chimeric polypeptide that includes (a) an extracellular domain (ECD) having a binding affinity for an antigen; (b) a hinge domain; (c) a transmembrane domain (TMD) derived from CD28; and (d) an intracellular signaling domain (ICD). In some embodiments, the hinge domain is capable of promoting dimerization of the chimeric polypeptide.

The nucleic acid constructs provided can contain naturally occurring sequences, or sequences that differ from those that occur naturally, but, due to the degeneracy of the genetic code, encode the same polypeptide, e.g., CAR. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid constructs are not limited to sequences that encode the chimeric polypeptides (e.g., CARs) of the disclosure; some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acids of the present disclosure can be introduced into a host cell to produce a recombinant cell containing the nucleic acid molecule. Accordingly, prokaryotic or eukaryotic cells that contain a nucleic acid encoding a chimeric polypeptide or a CAR as described herein are also features of the disclosure. In a related aspect, some embodiments disclosed herein relate to methods of transforming a cell that includes introducing into a host cell, such as an animal cell, a nucleic acid as provided herein, and then selecting or screening for a transformed cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In some embodiments, a nucleic acid of the disclosure are delivered by viral or non-viral delivery vehicles known in the art. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid is maintained and replicated in the recombinant host cell as an episomal unit. In some embodiments, the nucleic acid is stably integrated into the genome of the recombinant cell. Stable integration can be completed using classical random genomic recombination techniques or with more precise genome editing techniques such as using guide RNA directed CRISPR/Cas9 or TALEN genome editing. In some embodiments, the nucleic acid present in the recombinant host cell as a mini-circle expression vector for a stable or transient expression.

In some embodiments, the nucleic acids of the disclosure can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

Host cell useful in the present disclosure is one into which a nucleic acid construct as described herein can be introduced. Common host cells are mammalian host cells, such as, for example, HeLa cells (ATCC Accession No. CCL 2), HeLa S3 (ATCC Accession No. CCL 2.2), the African Green Monkey cells designated BSC-40 cells, which are derived from BSC-1 cells (ATCC Accession No. CCL 26), and HEp-2 cells (ATCC Accession No. CCL 23). In some embodiments, the host cells are Jurkat cells derivatives thereof. This is because Jurkat cell is an immortalized line of human T lymphocyte cells that can be suitably used, e.g. as a substitute for T cells, to study acute T cell leukemia, T cell signaling, and the expression of various chemokine receptors susceptible to viral entry, particularly HIV. Jurkat cells can produce interleukin 2, and can be used in research involving the susceptibility of cancers to drugs and radiation.

In some embodiments, the recombinant cell is a prokaryotic cell. In some embodiments, the recombinant cell is a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a human cell. In some embodiments, the cell is a non-human primate cell. In some embodiments, the recombinant cell is an immune system cell. e.g., a B cell, a monocyte, a NK cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor.

In some embodiments, the immune system cell is a T lymphocyte. In some embodiments, the lymphocyte is a T lymphocyte. In some embodiments, the lymphocyte is a T lymphocyte progenitor. In some embodiments, the T lymphocyte is a CD4+ T cell or a CD8+ T cell. In some embodiments, the T lymphocyte is a CD8+ T cytotoxic lymphocyte cell. Non-limiting examples of CD8+ T cytotoxic lymphocyte cell suitable for the compositions and methods disclosed herein include naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+ T helper lymphocyte cell. Suitable CD4+ T helper lymphocyte cells include, but are not limited to, naïve CD4+ T cells, central memory CD4+ T cells, effector memory CD4+ T cells, effector CD4+ T cells, CD4+ stem memory T cells, and bulk CD4+ T cells. In some embodiments, the Treg cells can be natural Tregs (nTregs) that include thymic Tregs (tTregs) and peripheral Tregs (pTregs), induced Tregs (iTregs), and engineering Tregs with forced expression of transgenes (such as IL-10, FOXP3), which in turn can confer suppressive functions.

In a related aspect, some embodiments of the disclosure relate to various methods for making a recombinant cell, including (a) providing a host cell capable of protein expression; and transducing the provided host cell with a nucleic acid construct of the disclosure to produce a recombinant cell. Non-limiting exemplary embodiments of the disclosed methods for making a recombinant cell can further include one or more of the following features. In some embodiments, the host cell is a T lymphocyte obtained by leukapheresis performed on a sample obtained from a subject, and the cell is transduced ex vivo. In some embodiments, the nucleic acid construct is encapsulated in a viral capsid or a lipid nanoparticle. In some embodiments, the methods further include isolating and/or purifying the produced cells. Accordingly, the recombinant cells produced by the methods disclosed herein are also within the scope of the disclosure.

Techniques for transforming a wide variety of the above-mentioned host cells and species are known in the art and described in the technical and scientific literature. For example, DNA vectors can be introduced into eukaryotic cells via conventional transformation or transfection techniques. Suitable methods for transforming or transfecting cells can be found in Sambrook, J., & Russell, D. W. (2012). Molecular Cloning: A Laboratory Manual (4th ed.) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection. In some embodiments, the nucleic acid molecule is introduced into a host cell by a transduction procedure, electroporation procedure, or a biolistic procedure. Accordingly, cell cultures including at least one recombinant cell as disclosed herein are also within the scope of this application. Methods and systems suitable for generating and maintaining cell cultures are known in the art.

### Pharmaceutical Compositions

The chimeric polypeptides, CARs, nucleic acids, recombinant cells, cell cultures of the disclosure can be incorporated into compositions, including pharmaceutical compositions. Such compositions generally include one or more of the chimeric polypeptides, CARs, nucleic acids, recombinant cells, cell cultures as provided and described herein, and a pharmaceutically acceptable excipient, e.g., carrier. In some embodiments, the pharmaceutical compositions of the disclosure are formulated for the prevention, treatment, or management of health conditions such as proliferative disorders (e.g., cancers).

In some embodiments, the composition includes one or more of the following: (a) one or more chimeric polypeptides as described herein; (b) one or more nucleic acid constructs as described herein; and (c) one or more recombinant cells as described herein. In some embodiments, the chimeric polypeptide, nucleic acid construct, or recombinant cell of the disclosure is formulated in a liposome. In some embodiments, the chimeric polypeptide, nucleic acid construct, or recombinant cell of the disclosure is formulated in a lipid nanoparticle. In some embodiments, the chimeric polypeptide, nucleic acid construct, or recombinant cell of the disclosure is formulated in a polymer nanoparticle.

In some embodiments, the composition includes one or more chimeric polypeptides as described herein and a pharmaceutically acceptable excipient. In some embodiments, the composition of the disclosure includes one or more recombinant cells as described herein and a pharmaceutically acceptable excipient. In some embodiments, the composition includes one or more nucleic acid constructs as described herein and a pharmaceutically acceptable excipient. In some embodiments, the nucleic acid construct is encapsulated in a viral capsid or a lipid nanoparticle.

In some embodiments, the composition of the disclosure is an immunogenic composition, e.g., a composition that can stimulate an immune response in a subject. In some embodiments, the immunogenic composition of the disclosure is formulated as a vaccine. In some embodiments, the immunogenic composition of the disclosure is formulated as an adjuvant.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the composition is formulated for one or more of intranasal administration, transdermal administration, intramuscular administration, intravenous administration, intraperitoneal administration, oral administration, or intra-cranial administration.

In some embodiments, the chimeric polypeptides and CARs of the disclosure can also be administered by transfection or infection using methods known in the art, including but not limited to the methods described in McCaffrey et al. (Nature 418:6893, 2002), Xia et al. (Nature Biotechnol. 20:1006-10, 2002), or Putnam (Am. J. Health Syst. Pharm. 53:151-60, 1996, erratum at Am. J. Health Syst. Pharm. 53:325, 1996).

As described in greater detail below, in some embodiments, the recombinant cells of the disclosure can be formulated for administration to a subject using techniques known to the skilled artisan. For example, formulations comprising populations of recombinant cells can include pharmaceutically acceptable excipient(s). Excipients included in the formulations will have different purposes depending, for example, on the recombinant cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising recombinant cells can have been prepared and cultured in the absence of non-human components, e.g., in the absence of animal serum. A formulation can include one population of recombinant cells, or more than one, such as two, three, four, five, six or more populations of recombinant cells.

Formulations comprising population(s) of recombinant cells can be administered to a subject using modes and techniques known to the skilled artisan. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Devices useful for parenteral injection of infusion of the formulations can be used to effect such administration.

### Methods of the Disclosure

Administration of any one of the therapeutic compositions described herein, e.g., chimeric polypeptides, CARs, nucleic acids, recombinant cells, cell cultures, and/or pharmaceutical compositions, can be used in the diagnosis, prevention, and/or treatment of relevant conditions, such as proliferative diseases (e.g., cancer). As described in greater detail below, in some embodiments of the disclosure, the chimeric polypeptides, CARs, nucleic acids, recombinant cells, cell cultures, and/or pharmaceutical compositions as described herein can be used for methods of modulating T-cell activation in a subject in need thereof. In some embodiments, the chimeric polypeptides, CARs, nucleic acids, recombinant cells, cell cultures, and/or pharmaceutical compositions as described herein can be incorporated into therapies and therapeutic agents for use in methods of preventing and/or treating an individual who has, who is suspected of having, or who may be at high risk for developing one or more health conditions, such as proliferative diseases (e.g., cancers), autoimmune disorders, and infections. In some embodiments, the individual is a patient under the care of a physician.

Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Accordingly, in one aspect, some embodiments of the disclosure relate to methods for the prevention and/or treatment of a condition in a subject in need thereof, wherein the methods include administering to the subject a composition including one or more of: a chimeric polypeptide of the disclosure, a nucleic acid construct of the disclosure, a recombinant cell of the disclosure, and/or a pharmaceutical composition of the disclosure.

In some embodiments, the compositions described herein, e.g., polypeptides, CARs, nucleic acids, recombinant cells, cell cultures, and/or pharmaceutical compositions, can be used in methods of treating individual who have, who are suspected of having, or who may be at high risk for developing cancer. In some embodiments, the chimeric polypeptides, CARs, nucleic acids, recombinant cells, cell cultures, and/or pharmaceutical compositions described herein can be used to inhibit tumor growth or metastasis of a cancer in the treated subject relative to the tumor growth or metastasis in subjects who have not been administered one of the therapeutic compositions disclosed herein. In some embodiments, the chimeric polypeptides, CARs, nucleic acids, recombinant cells, cell cultures, and/or pharmaceutical compositions described herein can be used to stimulate immune responses against the tumor via inducing the production of interferon gamma (IFNγ) and/or interleukin-2 (IL-2) and other pro-inflammatory cytokines. In some embodiments, the chimeric polypeptides, CARs, nucleic acids, recombinant cells, cell cultures, and/or pharmaceutical compositions described herein can be used to increases CAR-T cell survival in the subject and/or stimulate proliferation and/or killing capacity of CAR T-cells in the treated subject relative to the production of these molecules in subjects who have not been administered one of the therapeutic compositions disclosed herein. In some embodiments, the administered composition reduces CAR-T cell exhaustion in the subject. In some embodiments, the administered composition reduces CAR-T cell toxicity in the subject.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit the proliferation of the target cell and/or inhibit tumor growth of a target cancer in the subject compared to the proliferation of the target cell and/or tumor growth of the target cancer in subjects who have not been administered with the recombinant cells.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

Administration of the compositions described herein, e.g., polypeptides, CARs, nucleic acids, recombinant cells, cell cultures, and/or pharmaceutical compositions, can be used in the stimulation of an immune response.

An effective amount of the compositions described herein, e.g., chimeric polypeptides, CARs, nucleic acids, recombinant cells, cell cultures, and/or pharmaceutical compositions, is determined based on the intended goal, for example tumor regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each individual. Frequency of administration could range from 1-2 days, to 2-6 hours, to 6-10 hours, to 1-2 weeks or longer depending on the judgment of the practitioner.

Longer intervals between administration and lower amounts of compositions may be employed where the goal is prevention. For instance, amounts of compositions administered per dose may be 50% of the dose administered in treatment of active disease, and administration may be at weekly intervals. One of ordinary skill in the art, in light of this disclosure, would be able to determine an effective amount of compositions and frequency of administration. This determination would, in part, be dependent on the particular clinical circumstances that are present (e.g., type of cancer, severity of cancer).

In certain embodiments, it may be desirable to provide a continuous supply of a composition disclosed herein to the subject to be treated, e.g., a patient. In some embodiments, continuous perfusion of the region of interest (such as the tumor) may be suitable. The time period for perfusion would be selected by the clinician for the particular subject and situation, but times could range from about 1-2 hours, to 2-6 hours, to about 6-10 hours, to about 10-24 hours, to about 1-2 days, to about 1-2 weeks or longer. Generally, the dose of the composition via continuous perfusion will be equivalent to that given by single or multiple injections, adjusted for the period of time over which the doses are administered.

In some embodiments, administration is by bolus injection. In some embodiments, administration is by intravenous infusion. In some embodiments, a composition is administered is administered in a dosage of about 100 ng/kg of body weight per day to about 100 mg/kg of body weight per day. In some embodiments, a composition as disclosed herein is administered in a dosage of about 0.001 mg/kg to 100 mg/kg of body weight per day. In some embodiments, the therapeutic agents are administered in a single administration. In some embodiments, therapeutic agents are administered in multiple administrations, (e.g., once or more per week for one or more weeks). In some embodiments, doses are administered about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days. In some embodiments, there are 1, 2, 3, 4, 5, 6, 7, 8, 9, 10, or more total doses. In some embodiments, 4 doses are administered, with a 3 week span between doses.

One of ordinary skill in the art would be familiar with techniques for administering compositions of the disclosure to an individual. Furthermore, one of ordinary skill in the art would be familiar with techniques and pharmaceutical reagents necessary for preparation of these compositions prior to administration to an individual.

In certain embodiments of the present disclosure, the composition of the disclosure will be an aqueous composition that includes one or more of the chimeric polypeptides, CARs, nucleic acids, recombinant cells, cell cultures, and/or pharmaceutical compositions as described herein. Aqueous compositions of the present disclosure contain an effective amount of a composition disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the recombinant cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

One of ordinary skill in the art would appreciate that biological materials should be extensively dialyzed to remove undesired small molecular weight molecules and/or lyophilized for more ready formulation into a desired vehicle, where appropriate. The compositions described herein, e.g., chimeric polypeptides, CARs, nucleic acids, recombinant cells, cell cultures, and/or pharmaceutical compositions, will then generally be formulated for administration by any known route, such as parenteral administration. Determination of the amount of compositions to be administered will be made by one of skill in the art, and will in part be dependent on the extent and severity of cancer, and whether the recombinant cells are being administered for treatment of existing cancer or prevention of cancer. The preparation of the compositions containing the chimeric polypeptides, CARs, nucleic acids, recombinant cells, cell cultures, and/or pharmaceutical compositions of the disclosure will be known to those of skill in the art in light of the present disclosure.

Upon formulation, the compositions of the disclosure will be administered in a manner compatible with the dosage formulation and in such amount as is therapeutically effective. The compositions can be administered in a variety of dosage forms, such as the type of injectable solutions described above. For parenteral administration, the compositions disclosed herein should be suitably buffered. As discussed in greater detail below, the compositions as described herein may be administered with other therapeutic agents that are part of the therapeutic regiment of the individual, such as other immunotherapy or chemotherapy.

### Administration of Recombinant Cells to a Subject

In some embodiments, the methods of the disclosure involve administering an effective amount or number of the recombinants cells provided here to a subject in need thereof. This administering step can be accomplished using any method of implantation delivery in the art. For example, the recombinant cells can be infused directly in the subject's bloodstream or otherwise administered to the subject.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, i.e., long-term engraftment.

When provided prophylactically, the recombinant cells described herein can be administered to a subject in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to a subject in need thereof.

In some embodiments, the delivery of a recombinant cell composition (e.g., a composition including a plurality of recombinant cells according to any of the cells described herein) into a subject by a method or route results in at least partial localization of the cell composition at a desired site. A composition including recombinant cells can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection. For example, a population of recombinant cells are administered other than directly into a target site, tissue, or organ, such that it enters, the subject's circulatory system and, thus, is subject to metabolism and other similar biological processes.

The efficacy of a treatment including any of the compositions provided herein for the prevention or treatment of a disease or condition can be determined by a skilled clinician. However, one skilled in the art will appreciate that a prevention or treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

Measurement of the degree of efficacy is based on parameters selected with regard to the disease being treated and the symptoms experienced. In general, a parameter is selected that is known or accepted as correlating with the degree or severity of the disease, such as a parameter accepted or used in the medical community. For example, in the treatment of a solid cancer, suitable parameters can include reduction in the number and/or size of metastases, number of months of progression-free survival, overall survival, stage or grade of the disease, the rate of disease progression, the reduction in diagnostic biomarkers (for example without limitation, a reduction in circulating tumor DNA or RNA, a reduction in circulating cell-free tumor DNA or RNA, and the like), and combinations thereof. It will be understood that the effective dose and the degree of efficacy will generally be determined with relation to a single subject and/or a group or population of subjects. Therapeutic methods of the disclosure reduce symptoms and/or disease severity and/or disease biomarkers by at least about 1, 2, 3, 4, 5, 10, 15, 20, 25, 30, 35, 40, 45, 50, 55, 60, 65, 70, 75, 80, 85, 90, 95, 96, 97, 98, 99, or 100%.

As discussed above, a therapeutically effective amount includes an amount of a therapeutic composition that is sufficient to promote a particular beneficial effect when administered to a subject, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

### Additional Therapies

As discussed above, any one of the compositions as disclosed herein, e.g., the chimeric receptors, recombinant nucleic acids, recombinant cells, cell cultures, and pharmaceutical compositions described herein can be administered to a subject in need thereof as a single therapy (e.g., monotherapy) or as a first therapy in combination with at least one additional therapies (e.g., a second therapy), for example, with one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies. Accordingly, in some embodiments of the disclosure, the second therapy is selected from the group consisting of chemotherapy, radiotherapy, immunotherapy, hormonal therapy, toxin therapy or surgery.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

The present disclosure also contemplates the combination of the composition of the disclosure with other drugs and/or in addition to other treatment regimens or modalities such as surgery. When the composition of the present disclosure is used in combination with known therapeutic agents the combination may be administered either in sequence (either continuously or broken up by periods of no treatment) or concurrently or as an admixture. In the case of, for example, autoimmune diseases, treatment includes administering to the subject the compositions embodied herein, e.g. autologous T cells transduced or contacted with a CAR embodied herein and one or more anti-inflammatory agents and/or therapeutic agents. The anti-inflammatory agents include one or more antibodies which specifically bind to pro-inflammatory cytokines, e.g., pro-inflammatory cytokines such as interleukin-1 (IL-1), tumor necrosis factor alpha (TNFα), interleukin-6 (IL-6), granulocyte-macrophage colony-stimulating factor (GM-CSF), and interferon gamma (IFN-γ). In some embodiments, the antibodies are anti-TNFα, anti-IL-6 or combinations thereof. In some embodiments, one or more agents, other than antibodies can be administered which decrease pro-inflammatory cytokines, e.g. non-steroidal anti-inflammatory drugs (NSAIDs). Any combination of antibodies and one or more agents can be administered which decrease pro-inflammatory cytokines.

Treatment in combination is also contemplated to encompass the treatment with either the composition of the disclosure followed by a known treatment, or treatment with a known agent followed by treatment with the composition of the disclosure, for example, as maintenance therapy. For example, in the treatment of autoimmune diseases, excessive and prolonged activation of immune cells, such as T and B lymphocytes, and overexpression of the master pro-inflammatory cytokine tumor necrosis factor alpha (TNFα), together with other mediators such as interleukin-6 (IL-6), interleukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying anti-rheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pepol) have markedly improved the outcome of the management of autoimmune inflammatory diseases.

Non-steroidal anti-inflammatory drugs have the analgesic, antipyretic, and anti-inflammatory effect, frequently used for the treatment of conditions like arthritis and headaches. NSAIDs relieve pain through blocking cyclooxygenase (COX) enzymes. COX promotes the production of prostaglandins, a mediator which causes inflammation and pain. Although NSAIDs have different chemical structures, all of them have the similar therapeutic effect, e.g., inhibition of autoimmune inflammatory responses. In general, NSAIDs can be divided into two broad categories: traditional non-selective NSAIDs and selective cyclooxygenase-2 (COX-2) inhibitors (For a review, see, P. Li et al., Front Pharmacol (2017) 8:460).

In addition to anti-TNF agents, the biologics targeting other proinflammatory cytokines or immune competent molecules have also been extensively studied and actively developed. For example, abatacept, a fully humanized fusion protein of extracellular domain of CTLA-4 and Fc fraction of IgG1, has been approved for the RA patients with inadequate response to anti-TNF therapy. The major immunological mechanism of abatacept is selective inhibition of co-stimulation pathway (CD80 and CD86) and activation of T cells. Tocilizumab, a humanized anti-IL-6 receptor monoclonal antibody was approved for RA patients intolerant to DMARDs and/or anti-TNF biologics. This therapeutic mAb blocks the transmembrane signaling of IL-6 through binding with soluble and membrane forms of IL-6 receptor. Biological drugs targeting IL-1 (anakinra), Th1 immune responses (IL-12/IL-23, ustekinumab), Th17 immune responses (IL-17, secukinumab) and CD20 (rituximab) have also been approved for the treatment of autoimmune diseases (For a review see, P. Li et al., Front Pharmacol (2017) 8:460).

In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein. In particular, some embodiments of the disclosure provide kits for use in methods of modulating T-cell activation in a subject. Some other embodiments relate to kits for use in methods of preventing a health condition in a subject in need thereof. Some other embodiments relate to kits for use in methods of treating a health condition in a subject in need thereof. For example, provided herein, in some embodiments, are kits that include one or more of the chimeric polypeptides, CARs, nucleic acids, recombinant cells, cell cultures, and pharmaceutical compositions as provided and described herein, as well as written instructions for using the same.

In some embodiments, provided herein are kits that include one or more of the chimeric polypeptides as described herein, as well as written instructions for using the same in practicing a method described herein. In some embodiments, provided herein are kits that include one or more of the CARs as described herein, as well as written instructions for using the same in practicing a method described herein. In some embodiments, provided herein are kits that include one or more of the nucleic acids as provided and described herein, as well as written instructions for using the same in practicing a method described herein. In some embodiments, provided herein are kits that include one or more of the recombinant cells as described herein, as well as written instructions for using the same in practicing a method described herein. In some embodiments, provided herein are kits that include one or more of the pharmaceutical compositions as described herein, as well as written instructions for using the same in practicing a method described herein.

In some embodiments, the kits of the disclosure further include one or more means useful for the administration of any one of the provided chimeric polypeptides, nucleic acids, recombinant cells, cell cultures, and pharmaceutical compositions to a subject. For example, in some embodiments, the kits of the disclosure further include one or more syringes (including pre-filled syringes) and/or catheters (including pre-filled syringes) used to administer any one of the provided chimeric polypeptides, CARs, nucleic acids, recombinant cells, cell cultures, or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative controls, positive controls, reagents suitable for in vitro production and/or preparation of the chimeric polypeptides, CARs, nucleic acids, recombinant cells, or pharmaceutical compositions of the disclosure.

In some embodiments, the components of a kit can be in separate containers. In some other embodiments, the components of a kit can be combined in a single container. Accordingly, in some embodiments of the disclosure, the kit includes one or more of the compositions described herein, e.g., chimeric polypeptides, CARs, nucleic acids, recombinant cells, and pharmaceutical compositions of the disclosure in one container (e.g., in a sterile glass or plastic vial) and a further therapeutic agent in another container (e.g., in a sterile glass or plastic vial).

In another embodiment, the kit includes a combination of the compositions described herein, including chimeric polypeptides, CARs, nucleic acids, recombinant cells, and pharmaceutical compositions of the disclosure, in combination with one or more further therapeutic agents formulated together, optionally, in a pharmaceutical composition, in a single, common container.

In instances where the kit includes a pharmaceutical composition for parenteral administration to a subject, the kit can include a device (e.g., an injection device or catheter) for performing such administration. For example, the kit can include one or more needles (e.g., hypodermic needles) or other injection devices as discussed above containing one or more of the compositions described herein, e.g., chimeric polypeptides, CARs, nucleic acids, recombinant cells, and pharmaceutical compositions of the disclosure.

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Example 1

### General Experimental Procedures

### Human T Cell Isolation

Whole blood units were purchased from STEMCELL technologies (Vancouver, Canada). Peripheral blood mononuclear cells were isolated by Ficoll density gradient centrifugation and T cells were further enriched using the EasySep Human T Cell Isolation Kit (STEMCELL) per manufacturer's instructions. Enriched T cells were stained with antibodies against CD4, CD25, and CD127 and CD4⁺CD127⁺CD25^{low} conventional T cells were purified by fluorescence-activated cell sorting (FACS). Alternatively, enriched T cells were directly edited and activated with anti-CD3/CD28 beads and IL-2 (300 IU/mL, Prometheus laboratories, Nestle Health Science, Lausanne Switzerland). Cells were either used fresh or cryopreserved in fetal calf serum (FCS) with 10% DMSO and used later after thawing. When frozen cells were used, cells were thawed and cultured overnight in 300 IU/mL recombinant human IL-2 before editing and cell activation.

### Genome Editing Using Ribonucleoprotein Complex

Ribonucleoprotein complexes were made by complexing CRISPR RNAs (crRNAs) and trans-activating crRNAs (tracrRNA) chemically synthesized (Integrated DNA Technologies (IDT), Coralville, IA) with recombinant Cas9 protein (QB3 Macrolab, UC Berkley, CA) as previously described. Guide RNA sequences used for gene editing were:
T cell receptor beta chain constant region (TRBC): CCCACCAGCTCAGCTCCACG (SEQ ID NO: 7);
TRAC: CAGGGTTCTGGATATCTGT (SEQ ID NO: 24)
CD19: CGAGGAACCTCTAGTGGTGA (SEQ ID NO: 8);
CD28: TTCAGGTTTACTCAAAAACG (SEQ ID NO: 9).

Lyophilized RNAs were resuspended at 160 μM in 10 mM Tris-HCl with 150 mM KCl and stored in aliquots at −80° C. The day of electroporation, crRNA and tracrRNA aliquots were thawed and mixed at a 1:1 volume and annealed for 30 minutes at 37° C. The 80 μM guide RNA complex was mixed at 37° C. with Cas9 NLS at a 2:1 gRNA to Cas9 molar ratio for another 15 minutes. The resulting ribonucleoprotein complex (RNP) was used for genome editing. To delete TCR or CD28 genes, 1×10⁶ T cells were mixed with appropriate RNP and electroporated using a Lonza 4D 96-well electroporation system (pulse code EH115). For generating CD19-variants of Raji cells, parent Raji cells (ATCC® CCL-86™, Manassas, VA) were electroporated (pulse code EH140) with ribonucleoprotein complex targeting CD19 and the CD19− negative fraction was purified by FACS.

### Activation and Lentiviral Transduction of CD4⁺ T Cells

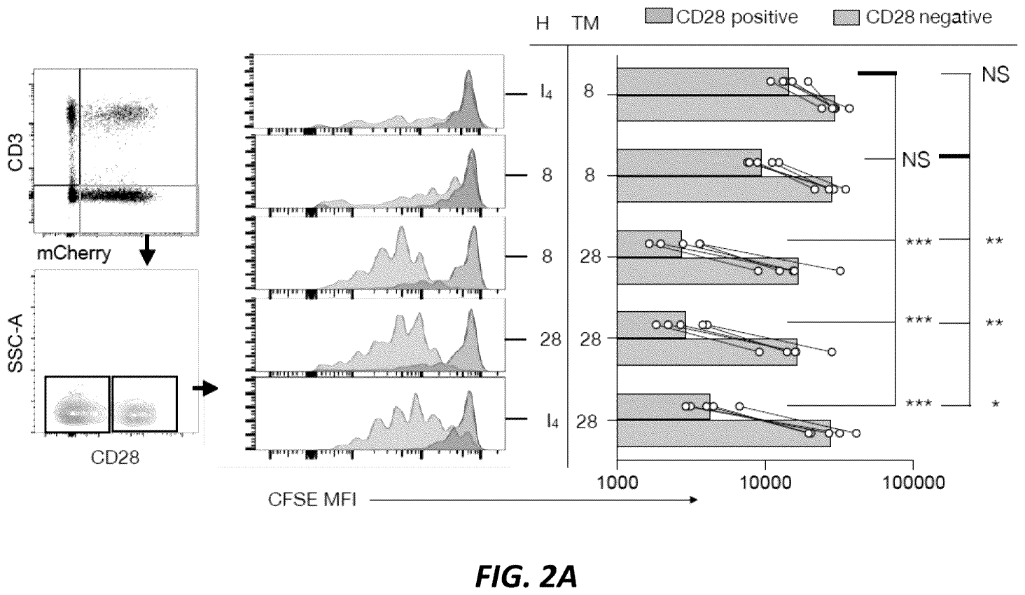

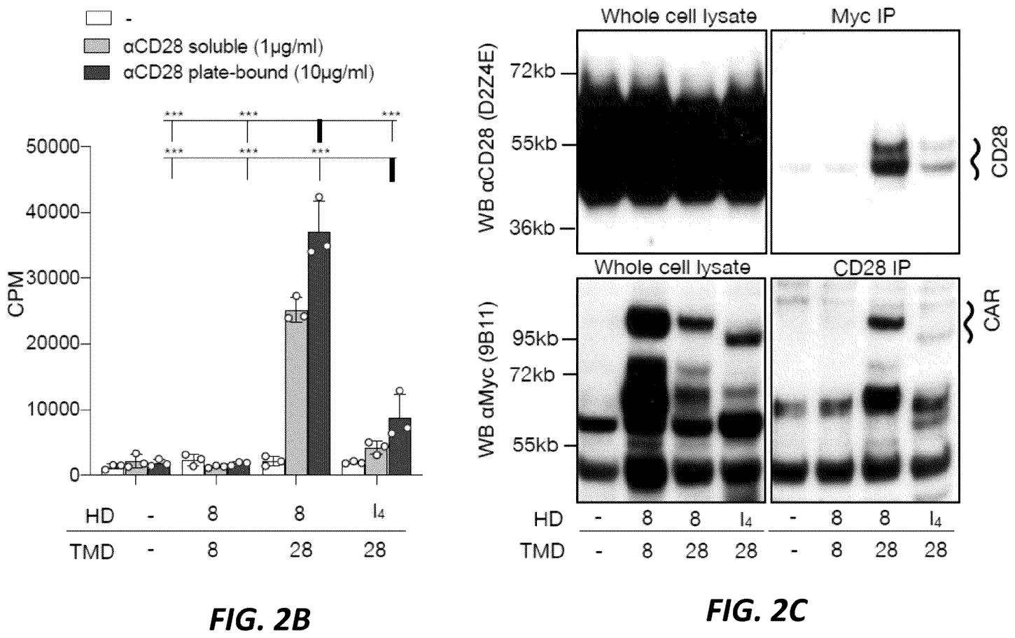

CD4⁺ T cells were electroporated prior to stimulation with anti-CD3/CD28 beads (Dynabeads Human T-Activator CD3/CD28, Thermo Fisher Scientific (Thermo Fisher), Waltham, MA). Cells were cultured in RPMI supplemented with 10% FCS and 300 IU/mL of IL-2 for the first two days after electroporation and reduced to 30 IU/mL of IL-2 for CD4 T cells and 100 IU/mL for bulk T cells thereafter. Lentiviruses encoding CD19-I4-28-4-1BBζ-T2A-EGFRt and CD19-I4-28-28ζ-T2A-EGFRt were from Juno Therapeutics (Bristol-Myers Squibb, New York, NY). Other lentiviral constructs, depicted in FIG. 2A, were cloned into the pCDH-EF1-FHC vector (Addgene plasmid #64874, Watertown, MA) as previously described. Briefly, genes encoding CAR constructs were purchased as gblocks (IDT) and amplified by PCR and cloned into the pCDH vector using Infusion cloning tools (Takara Bio, Kusatsu, Japan). Sequences for all clones used in subsequent experiments were confirmed by sequencing. All pLX302-based lentiviruses were produced and tittered by the viral core of UCSF. All lentiviruses were aliquoted and stored at −80° C. until use. Transduction was performed on day 2 after CD4⁺ T cell activation at a multiplicity of infection of 1 by spinoculation (1200 g, 30 minutes, 30° C.) in medium supplemented with 10% FCS and 0.1 mg/mL of protamine. Regarding AAV production, helper plasmid 30 μg of pDGM6 (a gift from YY Chen, University of California, Los Angeles), 40 μg of pAAV helper, and 15 nmol PEI were utilized. AAV6 vector production was carried out by iodixanol gradient purification. After ultracentrifugation, the AAVs were extracted by puncture and further concentrated using 50 mL Amicon column (Millipore Sigma Burlington, MA) and directly titrated on primary human T cells. Transduction was performed on day 2 after T cell activation.

### In Vitro Activation of Gene-Edited CAR T Cells

For some experiments, day-9 cells were re-stimulated without separating edited and transduced cells. For proliferation assays, the cell mixtures were stained with 2.5 μM carboxyfluorescein diacetate succinimidyl ester (CFDA SE, Thermo Fisher, referred to as CFSE) before re-stimulation with anti-CD3/CD28 beads. For some other experiments, day-9 cells were separated by FACS to purify CD3⁺ and CD3⁻ T cells with or without CAR. For assessing early T cell activation, purified CAR T cells were stimulated with soluble anti-CD28 (clone CD28.2, 1 μg/mL, BD Pharmigen), parental CD19⁺ Raji cells, or CD19-deficient Raji cells for 2 days. In some cultures, CTLA-4 Ig (kindly provided by Dr. Vincenti, UCSF) was added at a concentration of 13.5 μg/mL. For measurements of proliferation, purified cells were stimulated with soluble anti-CD28 (clone CD28.2, 1 μg/mL, BD Pharmigen), plate-bound anti-CD28 (clone CD28.2, 10 μg/mL), or soluble anti-CD3 (clone HIT3α 2 μg/mL. BD Pharmigen). After 48 hours, a portion of the supernatant was collected and analyzed for cytokine secretion using multiplexed Luminex (Eve Technologies, Calgary, Canada). The cells were then pulsed with 0.5 μCi of 3H thymidine and cultured for another 16-18 hours before harvesting the cells to determine the level of 3H thymidine incorporation using a scintillation counter.

### Flow Cytometry

The following antibodies were used for phenotyping and proliferation assays: anti-CD3-PE/Cy7 (clone SK7, Biolegend, San Diego, CA), anti-CD4-PerCP (clone SK3, BD Pharmigen, San Jose, CA), anti-CD4 A700 (clone RPA T4, Biolegend), anti-CD19 APC (clone HIB19, BD Pharmigen), anti-CD25 APC (clone 2A3, BD Pharmigen), anti-CD71 FITC (clone CY1G4, Biolegend), anti Myc FITC or APC (clone 9B11, Cell Signaling, Danvers, MA), anti-FMC19 idiotype APC (Juno therapeutics), anti-EGFRt PE (Juno therapeutics), anti-CD28 APC (clone 28.2, Biolegend), CD8 APC-Cy7 (clone SKI Biolegend). DAPI (Thermo Fisher, Waltham, MA) was used to stain dead cells for exclusion during analysis. For in vitro mixed lymphocyte reactions or CFSE in vivo analysis, Fc-block (Sigma-Aldrich, St. Louis, MO) was used (20 μg/mL) 5 minutes prior to surface staining. Flow cytometric analyses were performed on an LSRII flow cytometer (BD Pharmigen). Fluorescence-activated cell sorting was performed on FACSAria III (BD Pharmigen). All flow cytometry data were analyzed using Flowjo software (Tree Star, Ashland, OR).

### Immunoprecipitation

FACS-purified CD3⁻CAR⁺ or CD3⁻CAR⁻ CD4⁺ T cells (8×10⁶ each) were lysed in Pierce™ IP Lysis Buffer (Thermo Fisher) supplemented with Complete Protease Inhibitor Cocktail (Roche, Basel, Switzerland) for 30 minutes using a vertical rotator. Cell lysis was completed by briefly sonicating cells using a Q500 sonicator (QSonica, Newtown, CT). Pierce™ anti-c-Myc magnetic beads (clone 9E10, Thermo Fisher) were used for immunoprecipitation of the CAR. Alternatively, rabbit anti-human CD28 (clone D2Z4E, Cell Signaling) followed by anti-rabbit IgG Pierce™ protein A/G magnetic beads (Thermo Fisher) were used for CD28 immunoprecipitation of the cell lysate according to the manufacturer's instructions.

### Western Blot

Equal masses of protein lysate or equal volumes of immunoprecipitation eluents were loaded into NUPAGE 4-12% Bis-Tris gels (Thermo Fisher). After electrophoresis, proteins were transferred onto PVDF membranes (Thermo Fisher) using an iBlot 2 Dry Blotting system. After blocking with Tris-buffered saline with 0.1% Tween-20 and 5% bovine serum albumin (TBSTB), membranes were stained with primary and secondary antibodies diluted in TBSTB. The following antibodies were used: mouse anti-Myc (clone 9B11, Cell Signaling), rabbit anti-CD28 (clone D2Z4E, Cell Signaling), HRP-conjugated anti-mouse IgG (Cell Signaling) and HRP-conjugated anti-rabbit IgG (Cell Signaling).

### Three Dimensional Model Prediction and Validation

Structural modeling of the different CARs was performed using Iterative Threading ASSembly Refinement (I-TASSER) software (Yang et al., Nat. Methods, 2015). Amino acid corresponding to the scFv was modeled on the UCHT1 scFv template (PDB ID code 1XIW) (Arnett et al. Proc Natl Acad Sci USA, 2004). The HD coordinates were recovered from the crystal structure of the pembrolizumab template (PDB ID code 5DK3) (Scapin et al., Nat Struct. Mol. Biol., 2015) and the crystal structure of human CD28 (PDB ID code 1YJD) (Evans et al., Nat. Immunol. 2005) for IgG4 and CD28, respectively. Modeling of the CD8-HD was performed using the Rosetta protein modeling suite (Leman et al., Nat. Methods, 2020). Structures were assembled with PyMOL (Schrodinger, LLC). Models were further evaluated with MolProbity software (Salter et al., Sci. Signal. (2018).

### Example 2

### Anti-CD28 Stimulation of CD19-CAR T Cells is TMD Dependent

This Example describes the results of experiments performed to demonstrate that anti-CD28 stimulation of CD19-CAR T cells is TMD dependent.

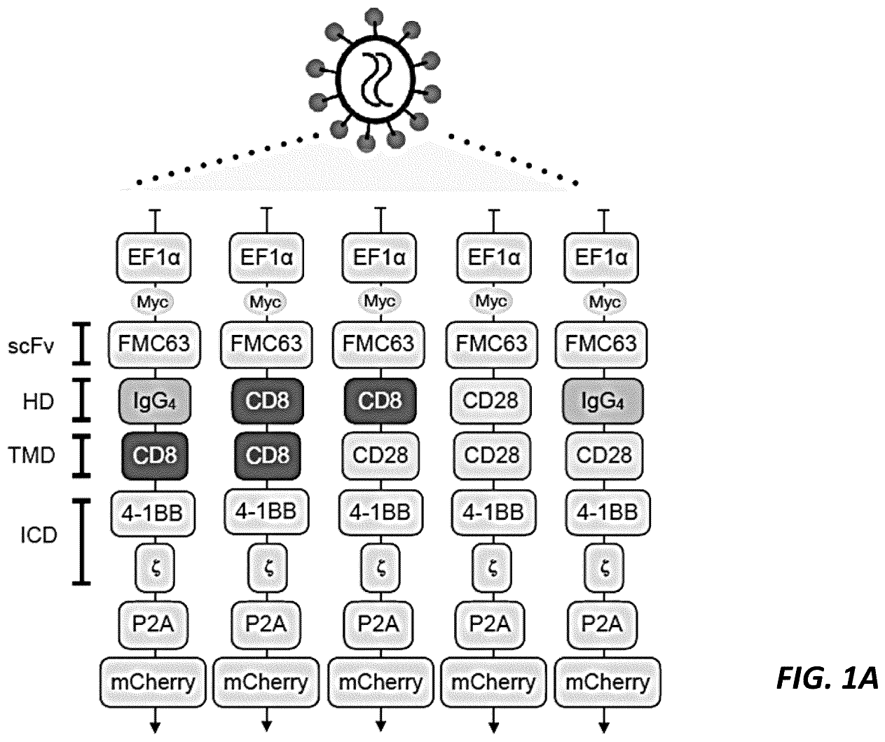

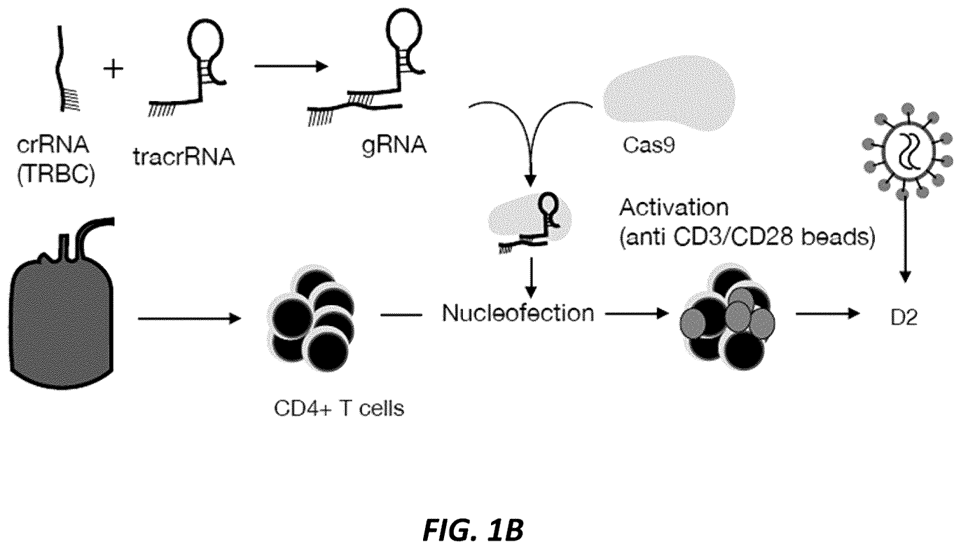

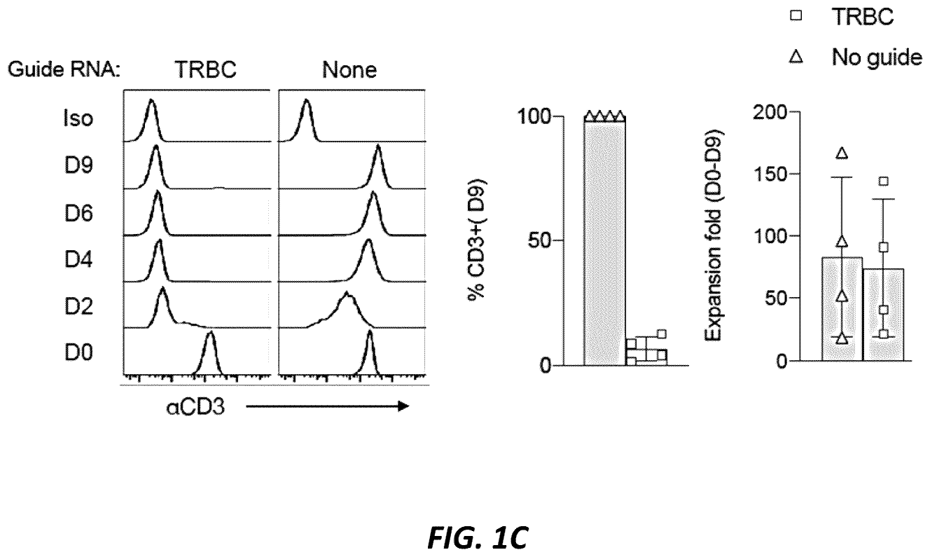

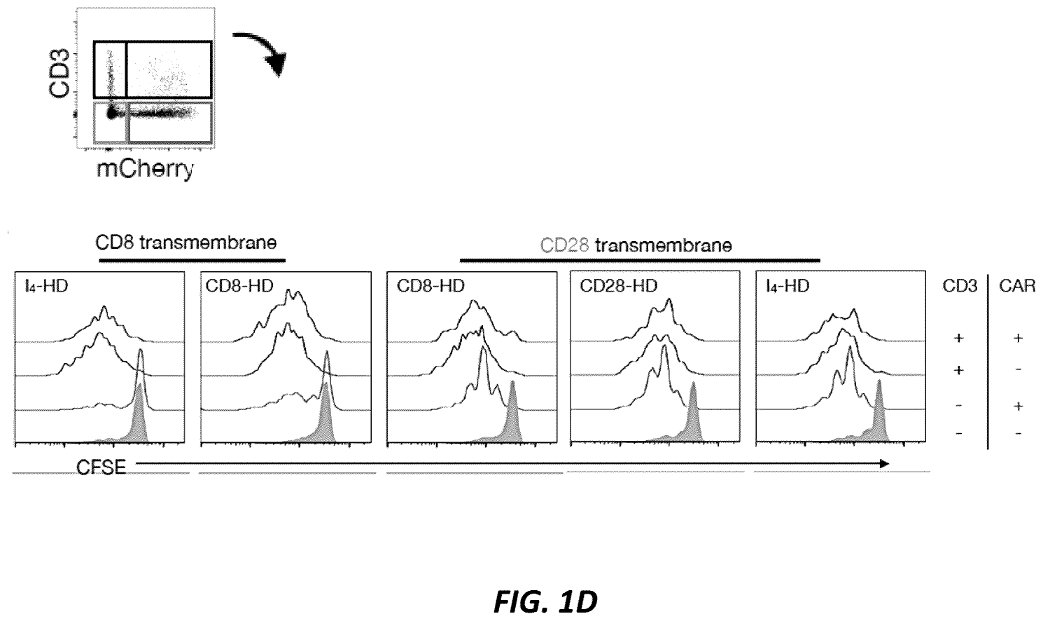

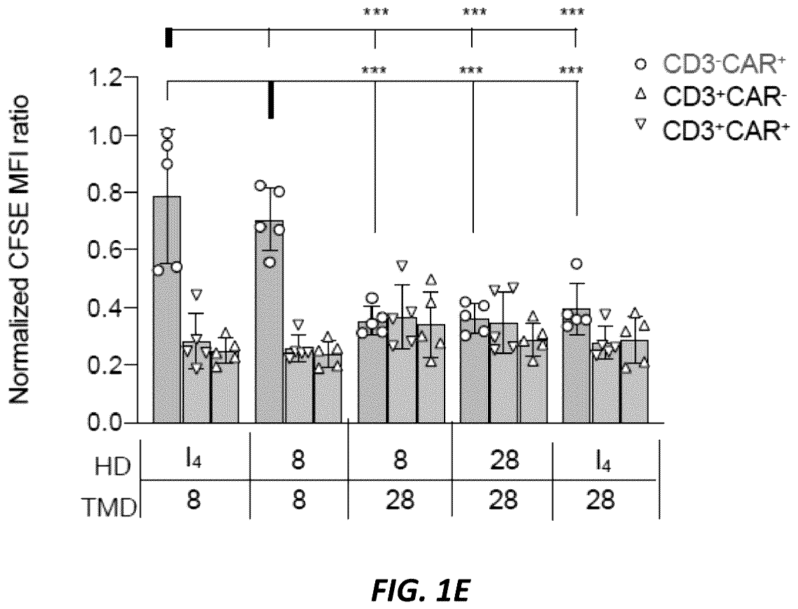

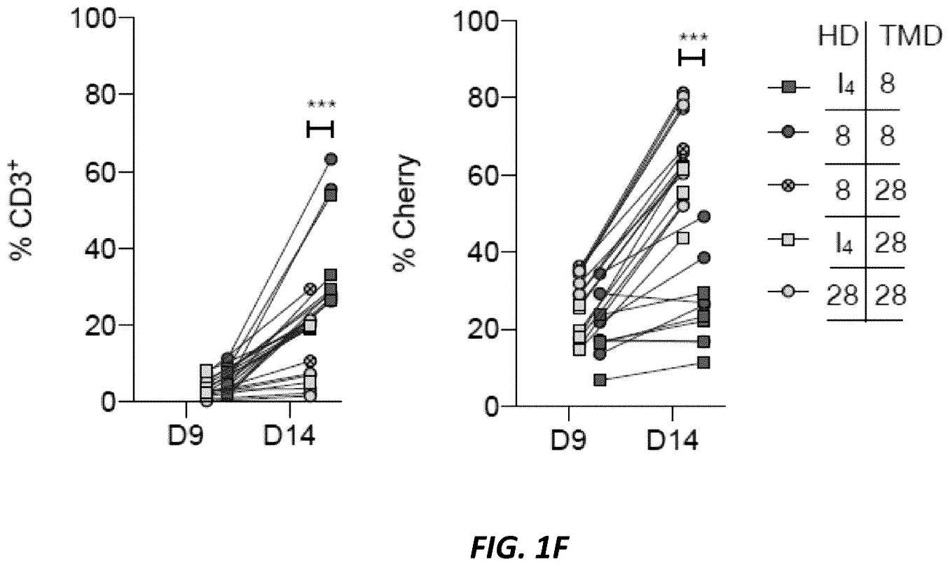

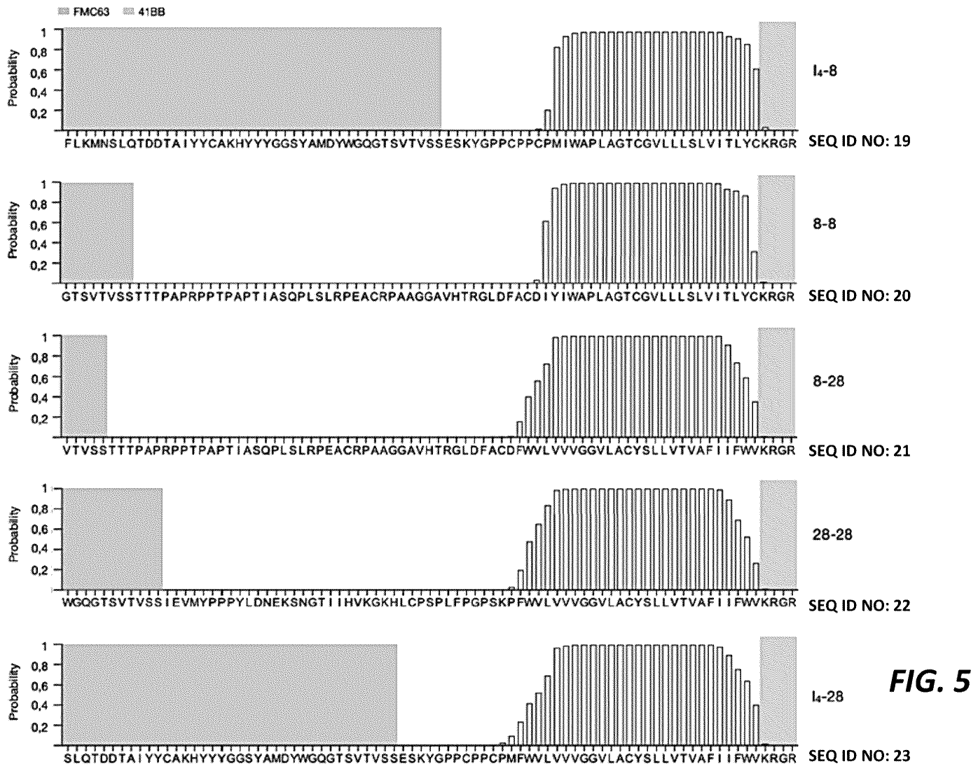

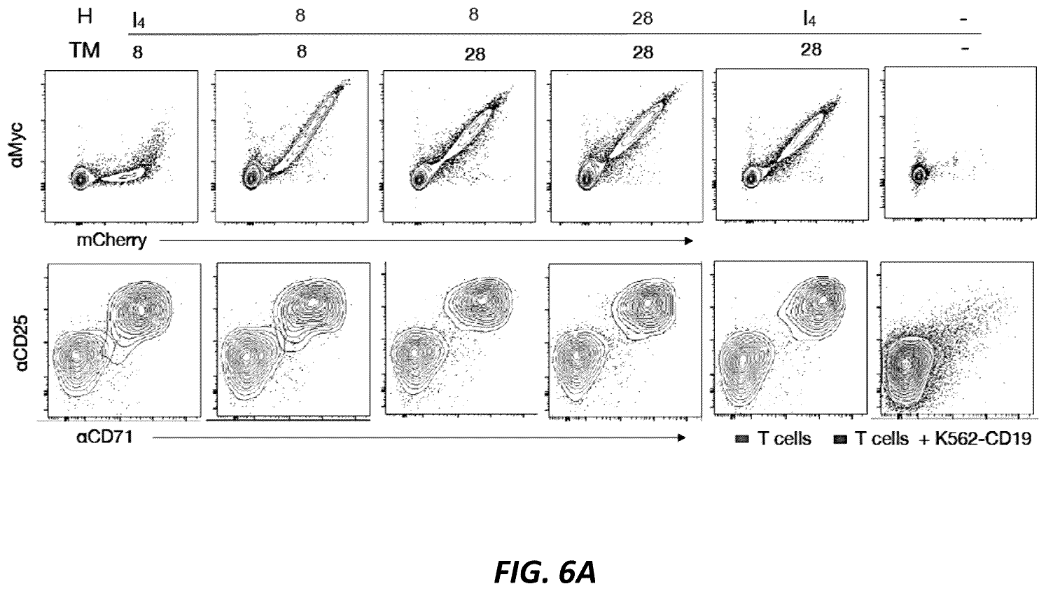

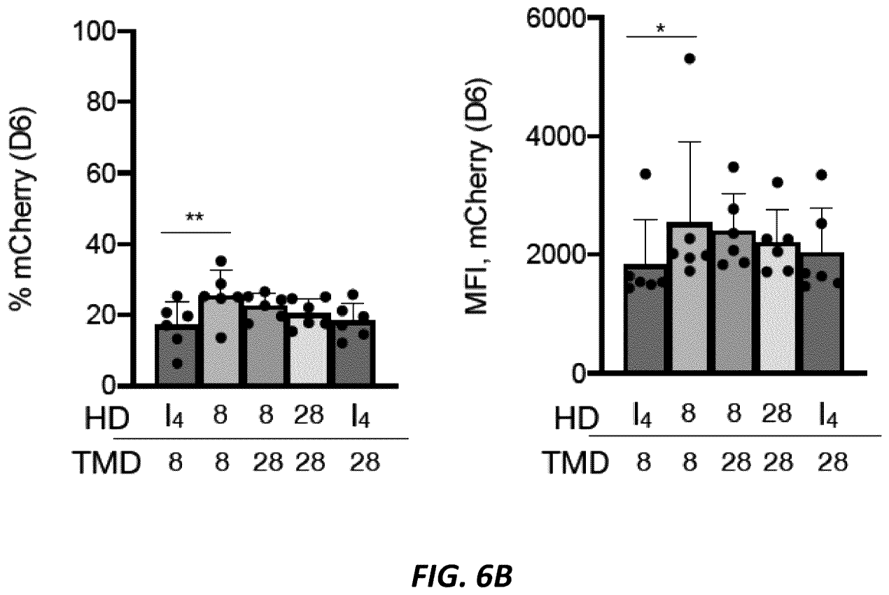

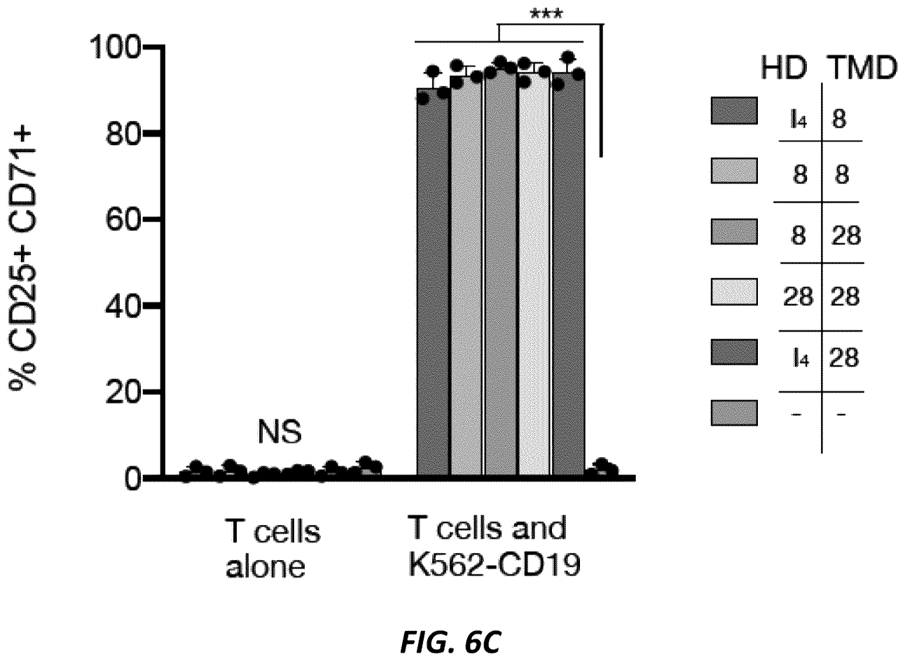

In these experiments, to investigate the role of CAR TMD, a panel of CD19-CARs was generated. These CD19-CARs differed from one another only by their hinge domain (HD derived from CD8, CD28 or IgG4) and TMD (CD8 versus CD28), all of which have been used to engineer CAR T cells for clinical applications (see, e.g., FIG. 1A and FIG. 5). Each CAR was designed with a MYC tag on the N-terminus of the scFv and a mCherry reporter (see, e.g., FIG. 1A). The 4-1BB's ICD was selected to avoid potential interactions with the endogenous CD28. Furthermore, the TCR beta chain constant region (TRBC) locus was disrupted using CRISPR/Cas9 to prevent any potential confounding influence by the endogenous TCR (see, e.g., FIG. 1B). The TRBC gene-disrupted T cells retained cell surface expression of TCR/CD3 proteins for a few days after editing and could thus be activated with anti-CD3/CD28 beads. Edited CD4⁺ T cells were transduced with various lentiviral CAR constructs by spinoculation two days after activation. On day 9 after stimulation, 87-98% of the cells were CD3− negative, demonstrating successful TCR deletion in the majority of the cells (see, e.g., FIG. 1C). Comparable transduction efficiencies were observed across the different CAR constructs, as assessed by mCherry expression and all CAR T cells responded to CD19 re-stimulation (see, e.g., FIG. 6).

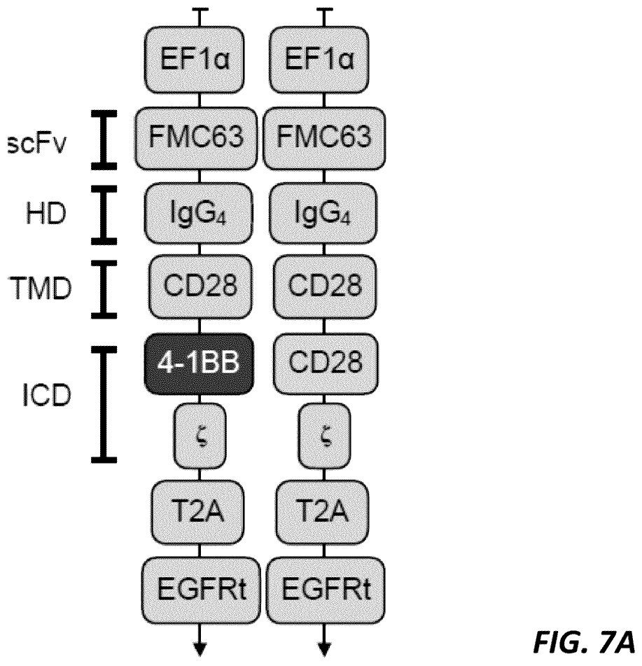

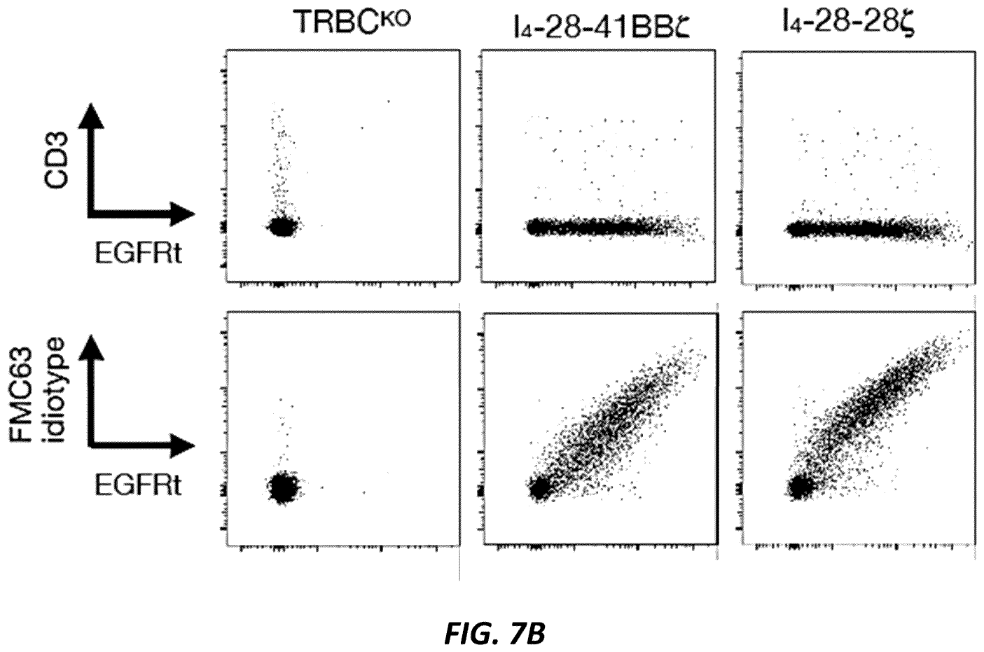

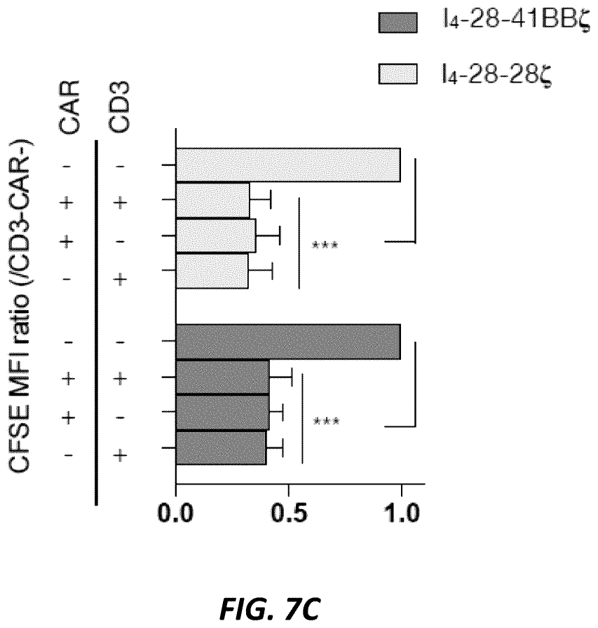

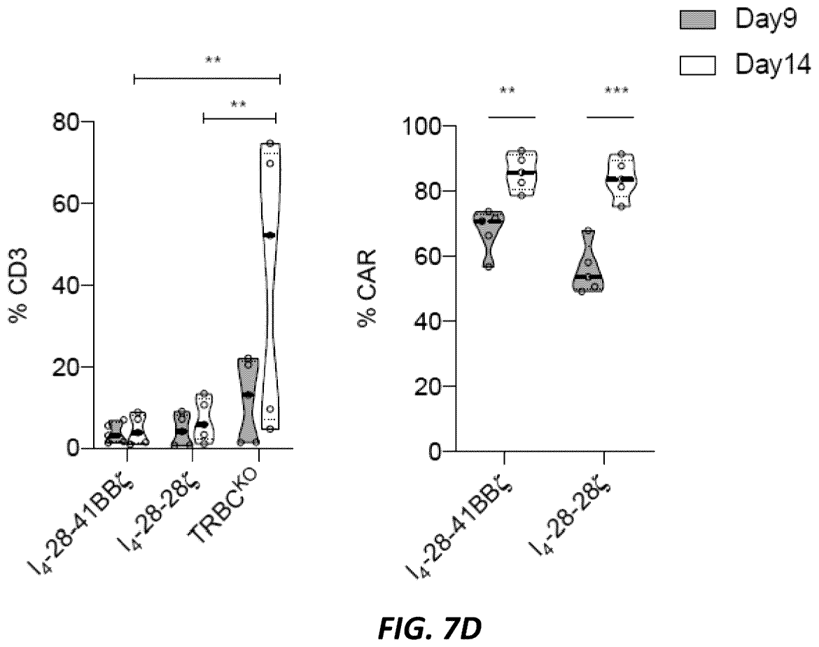

Re-stimulation of TCR-edited CAR-transduced T cells, containing a mixed population of CD3^{+/-} and CAR^{+/-} cells, with anti-CD3/CD28 beads on day 9, resulted in the expansion of CD3⁺ T cells that escaped TCR deletion (see, e.g., FIGS. 1D and 1E). Surprisingly, TCR-deficient, CD3⁻CAR⁺ T cells with CARs containing a CD28-TMD, but not CD8-TMD, also proliferated. Consequently, CAR⁺ T cells transduced with CARs containing a CD28-TMD, but not CD8-TMD, were enriched at the end of the 5-day re-stimulation (FIG. 1F). The lack of proliferation of CD8-TMD-containing CAR T cells suggests that expansion was not a consequence of bystander effects, such as IL-2 production by the CD3⁺CAR⁺ T cells in the same culture. To determine if this is unique to CARs with 4-1BB-ICD, the experiment was repeated using CARs with a CD28-ICD and observed a similar pattern of proliferation and enrichment of CD3⁻CAR⁺ T cells after anti-CD3/28 bead re-stimulation (FIGS. 7A-7D).

### Example 3

### CD28-TMD CARs Interact with Endogenous CD28 Receptor which is Required for Proliferation in Response to Anti-CD3/CD28

This Example describes the results of experiments performed to demonstrate that CD28-TMD CARs interact with endogenous CD28 receptor which is required for proliferation in response to anti-CD3/CD28. In these experiments, in order to verify that the endogenous CD28 receptor was required for proliferation in response to anti-CD3/CD28 beads, both the CD28 and TRBC genes were deleted in T cells before activation and lentiviral CAR transduction (FIG. 2A). CD3⁻CAR⁺CD28⁺ T cells expressing CARs containing a CD28-TMD, but not a CD8-TMD, proliferated in response to anti-CD3/28 beads. Deletion of CD28 abrogated the ability of CD28-TMD-containing CAR T cells to proliferate in response to anti-CD3/CD28 beads, demonstrating that CD28-mediated activation depends on endogenous CD28 expression (see, e.g., FIG. 2A).

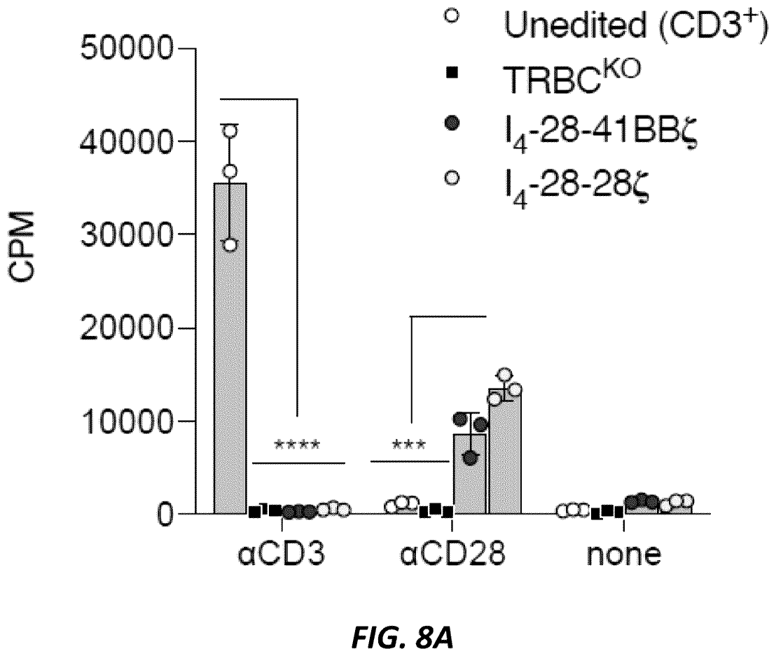

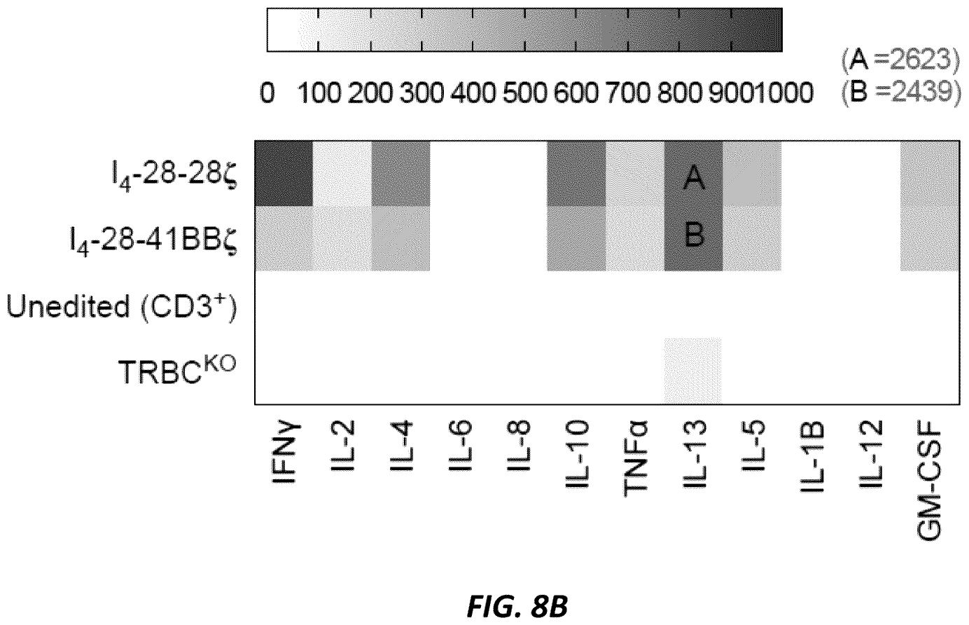

To determine if CD3⁻ T cells with a CD28-TMD-containing CAR can respond to anti-CD28 stimulation alone without influence from other cells in the culture, CD3⁻CAR⁺ cells were FACS-purified before re-stimulation with plate-bound or soluble anti-CD28 antibodies (clone CD28.2). For these experiments, CD28-HD containing CARs were excluded to avoid potential interaction mediated by the CD28-HD. The results confirmed that CAR T cells engineered with a CD28-TMD, but not a CD8-TMD, proliferated in response to anti-CD28 alone (see, e.g., FIG. 2B). The proliferative response induced by anti-CD28 alone in CD3⁻CAR⁺ T cells was similar in CAR T cells with a 4-1BB or a CD28 costimulatory domain in their ICD (see, e.g., FIG. 8A). Moreover, anti-CD28 induced secretion of multiple cytokines by CD3⁻CAR⁺ T cells, but not by CD3⁺CAR⁻ or CD3⁻CAR⁻ control cells (see, e.g., FIG. 8B). Collectively, these results show that CD28-TMD containing CARs can be activated by anti-CD28 without antigen recognition by the CAR or TCR. These results, together with recent reports of phosphorylation of endogenous CD28 upon CAR stimulation (with a CD28-TMD domain), suggest interactions between CD28 and CD28-TMD-containing CARs.

Additional co-immunoprecipitation experiments were performed to directly determine if CD28-TMD-containing CAR and CD28 physically interact. CD28-TMD-containing, but not CD8-TMD containing, CARs co-immunoprecipitated with endogenous CD28. Conversely, endogenous CD28 co-immunoprecipitated with CD28-TMD-containing, but not CD8-TMD-containing, CARs, demonstrating that the CD28-TMD of the CAR interacted with the endogenous CD28 receptor (see, e.g., FIG. 2C). It is worth noting that CD8-HD/CD28-TMD CARs and CD28 co-immunoprecipitated more efficiently when compared to the IgG4-HD-CD28-TMD construct, which is consistent with the better proliferation observed with CD8-HD/CD28-TMD CAR upon anti-CD28 stimulation (see, e.g., FIG. 2B). Without being bound to any particular theory, it is hypothesized that this difference may be due to the very short IgG4-HD (see, e.g., FIG. 5) which may not be as flexible as other hinges, leading to steric hindrance by the globular scFv domain. Alternatively, the membrane proximity of the cysteine in the IgG4-HD may not readily form disulfide bonds with the cysteine in the CD28 hinge of the endogenous CD28 receptor.

### Example 4

### Molecular Basis of CD28-TMD Dimerization

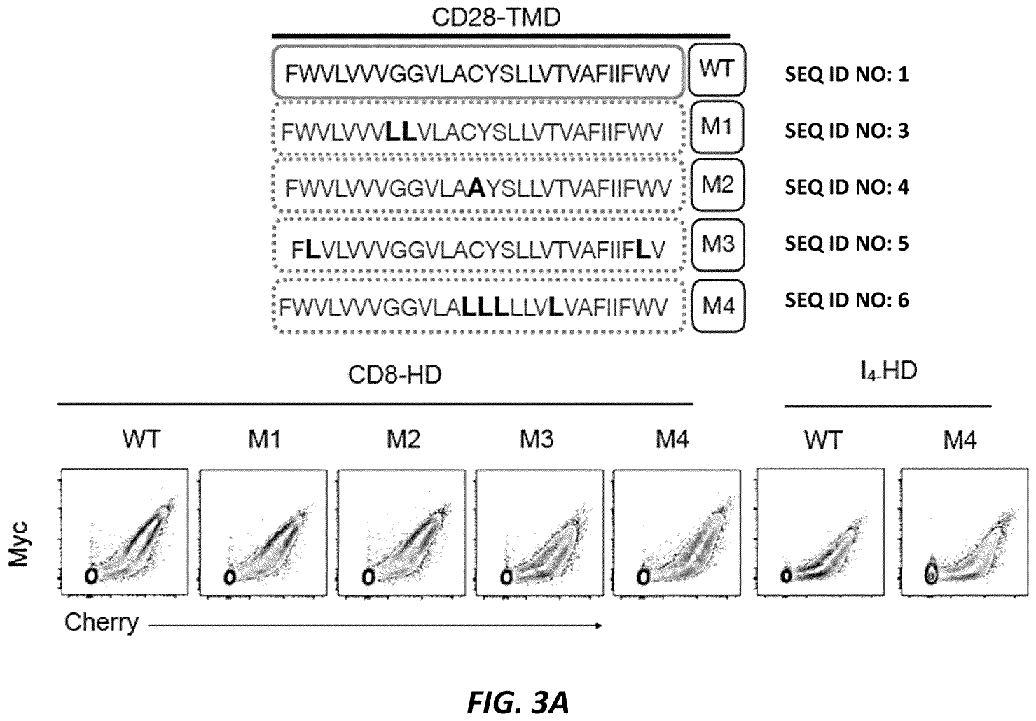

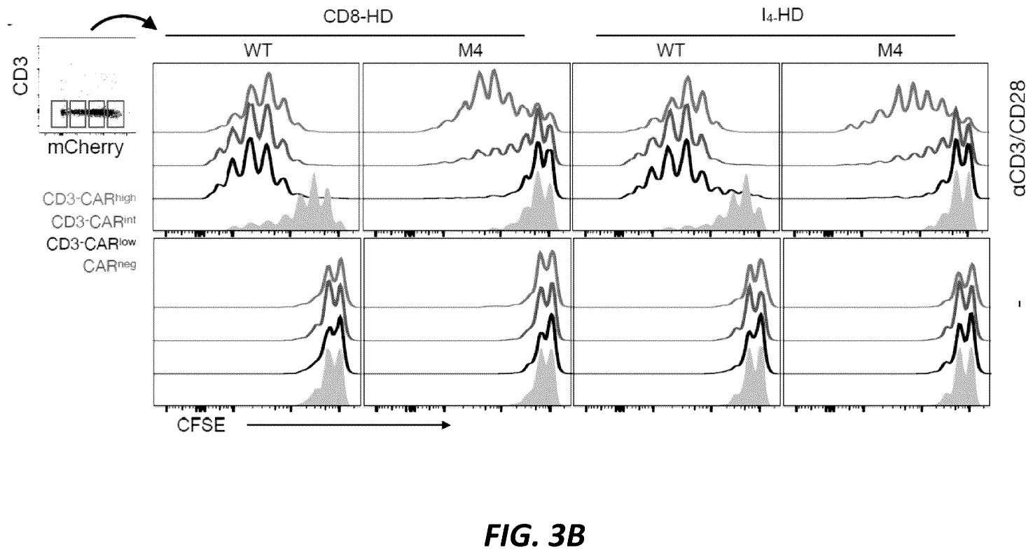

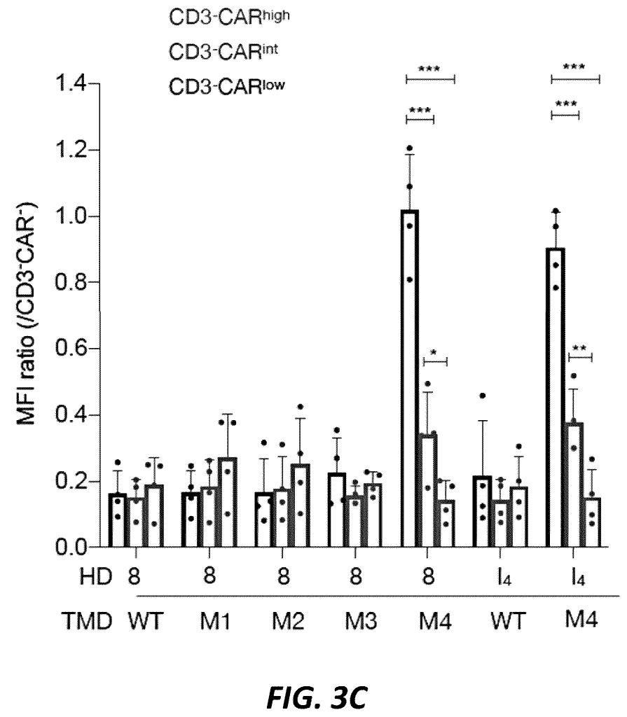

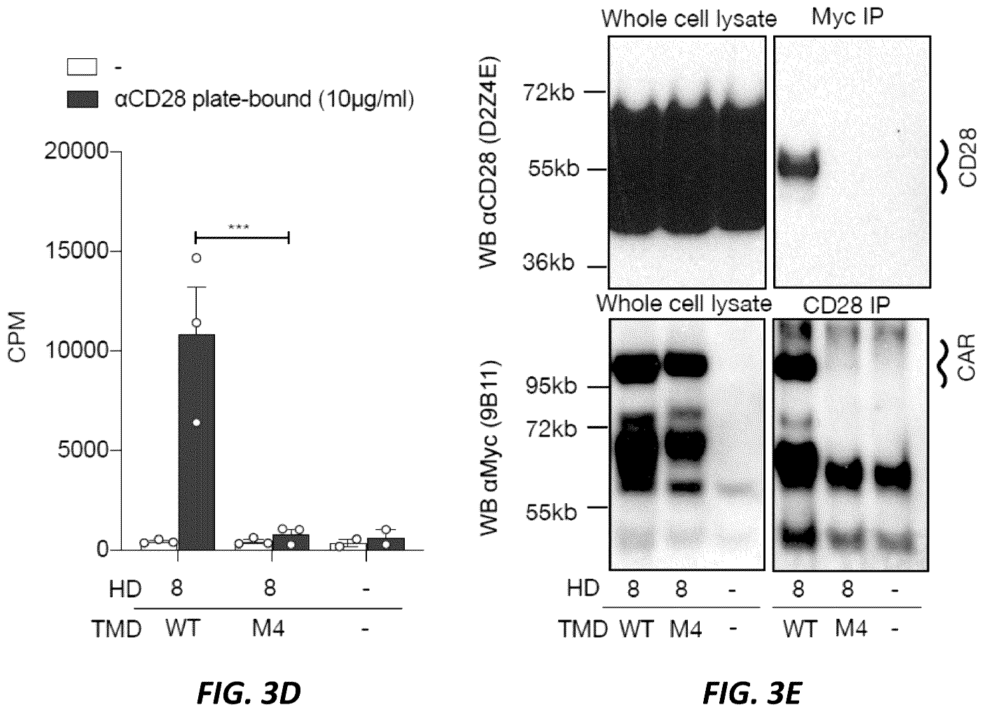

This Example describes the results of experiments performed to identify the molecular basis of CD28-TMD dimerization. In these experiments, a series of CD28-TMD CAR mutants was generated to determine the molecular basis of the CAR-CD28 interaction. The two glycine G160L and G161L (M1) (i.e., G8L and G9L of the CD28-TMD) that may function as part of a glycine-zipper motif, a process known to control TMD dimerization, were first mutated. The second mutation replaced the C165 cysteine to alanine (i.e., C13A of the CD28-TMD), as cysteine can form disulfide bonds (M2). The third (M3) and fourth (M4) sets of mutations were made on amino acids targeting either the two bulky hydrophobic tryptophans at the border of the TMD (W154L and W179L of human CD28 protein; corresponding to W2L and W26L of the CD28-TMD) or the four amino-acid residues in the core of the TMD (C165L, Y166L, S167L, and T171L), as cysteine could form a disulfide bond and others may hydrogen bond across the interface (see, e.g., FIG. 3A). All CARs with TMD-mutants were readily expressed on the cell surface (see, e.g., FIG. 3A). The various CD3⁻CAR⁺ cells with mutated CD28-TMD were examined for their ability to proliferate to anti-CD28 stimulation. Based on the level of mCherry expression, CD3⁻CAR⁺ cells were defined as low, intermediate, or high CAR expressors. CAR T cells with the wild type CD28-TMD (CD28^{WT}-TMD) proliferated to anti-CD3/CD28 stimulation regardless of the level of CAR expression (FIGS. 3B-3C). The CD28^{M4}-TMD, but not the other TMD mutants, abrogated the proliferation of CD3-CAR^{low} cells and significantly reduced proliferation of CD3-CAR^{int} cells with either CD8-HD or IgG4-HD (FIGS. 3B-3C). Interestingly, CD3⁻ CAR^{high} T cell proliferation was only weakly affected by M4 mutations. The proliferation of CD3⁻CAR^{high} T cells was not observed under conditions when the cells were not re-stimulated, demonstrating that activation was dependent on anti-CD28 stimulation, and not a result of autonomous CAR tonic signaling (see, e.g., FIG. 3C). It has been reported that CD28-mediated co-stimulation can induce coalescence of membrane microdomains enriched for signaling molecules resulting in enhanced T cell activation. Thus, cells expressing high levels of CAR may become sufficiently activated to proliferate by CD28-induced membrane compartmentalization.

To confirm that the CD28^{M4}-TMD disrupted the interaction between CD28 and the CAR, CAR T-cells engineered either with a CD8-HD/CD28^{WT}-TMD or with CD8-HD/CD28^{M4}-TMD were sorted and re-challenged with plate-bound anti-CD28 (see, e.g., FIG. 3D). In this assay, only CAR T cells with a CD28^{WT}-TMD showed significant proliferation, as measured by radiolabeled-thymidine incorporation. Importantly, co-immunoprecipitation of the endogenous CD28 and CD28-TMD-containing CAR was abrogated by the M4-mutant, demonstrating that the four amino acids in the core of the CD28-TMD are necessary for CAR-CD28 heterodimerization (see, e.g., FIG. 3E). Taken together, experimental data presented thus far demonstrate a previously unrecognized function of the CD28-TMD in mediating CAR interaction with endogenous CD28.

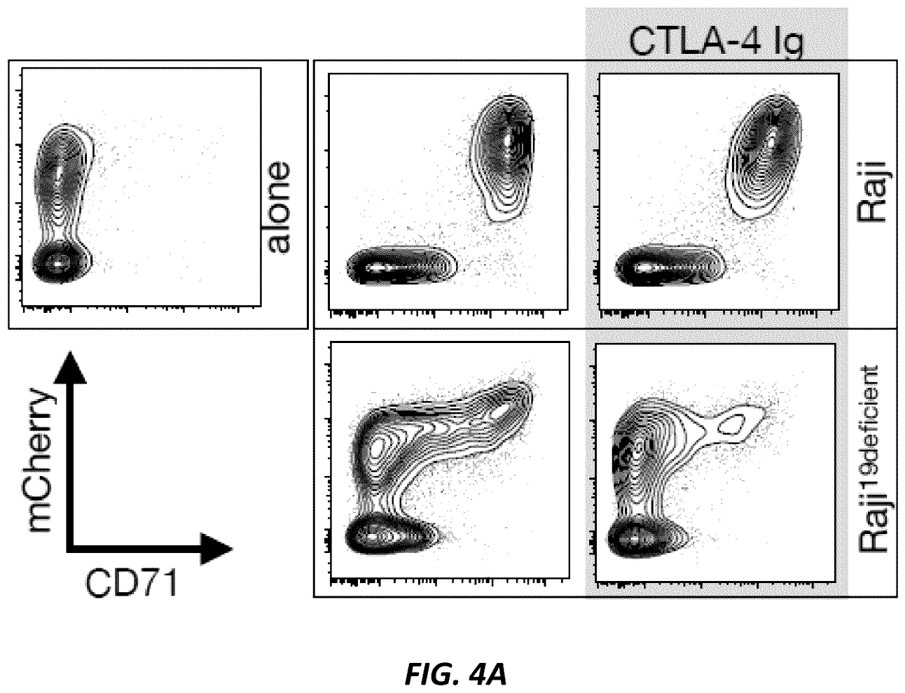

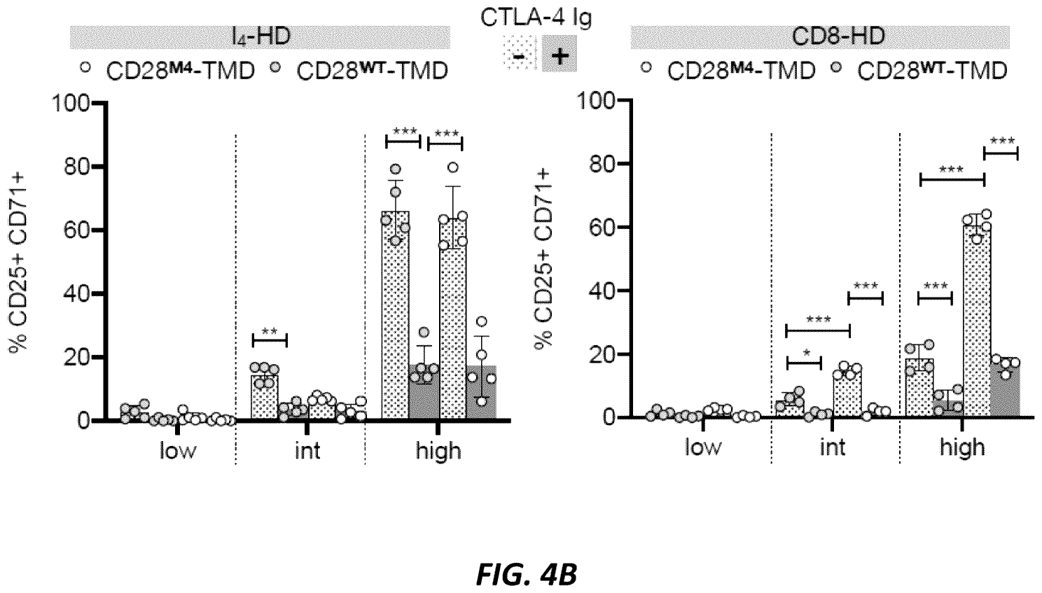

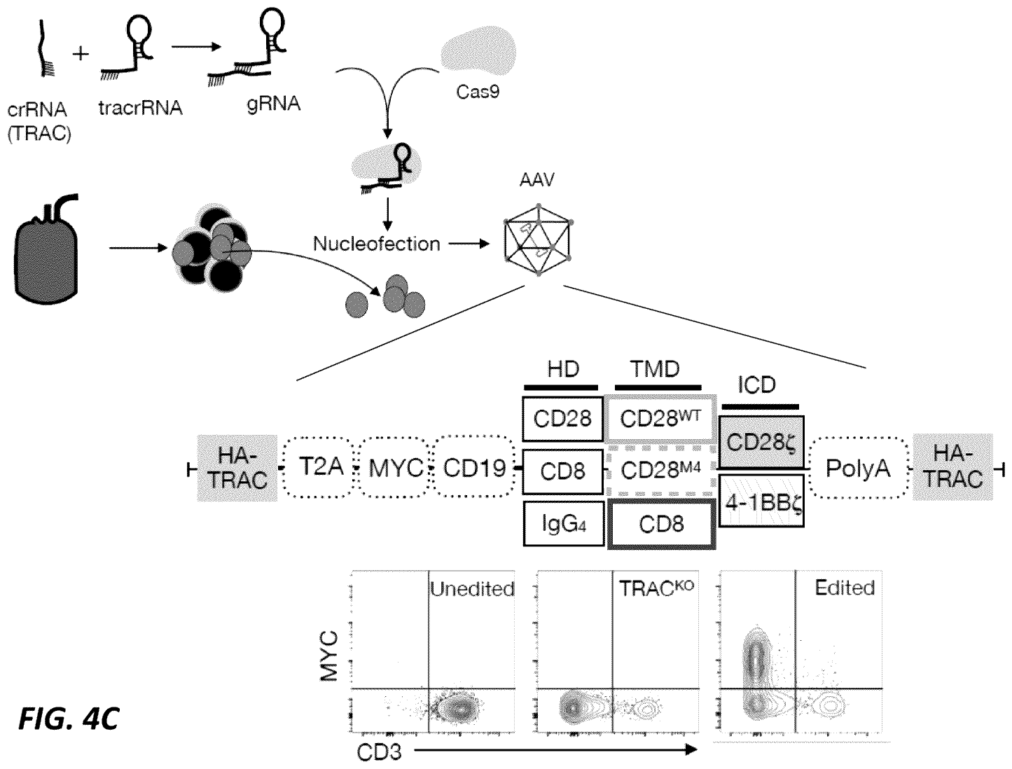

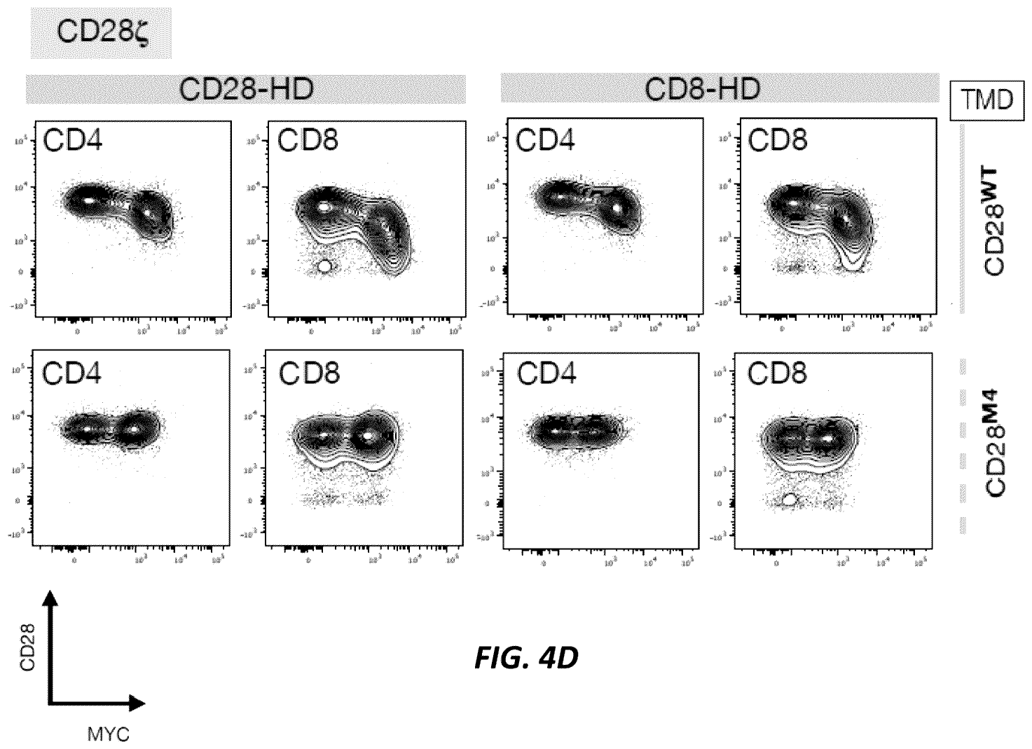

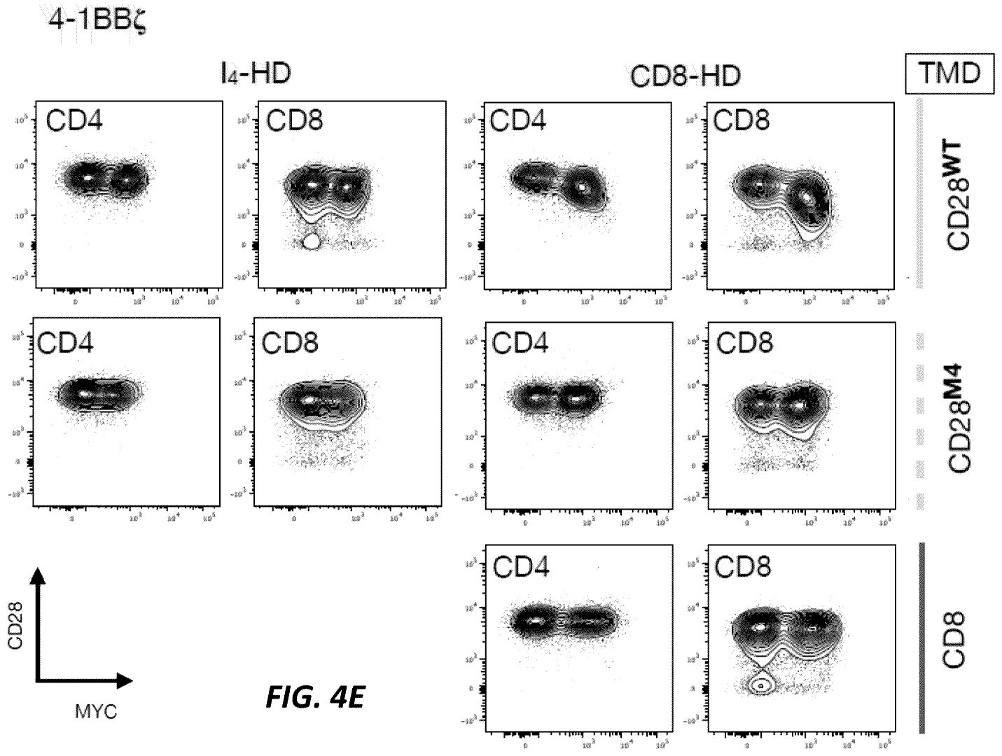

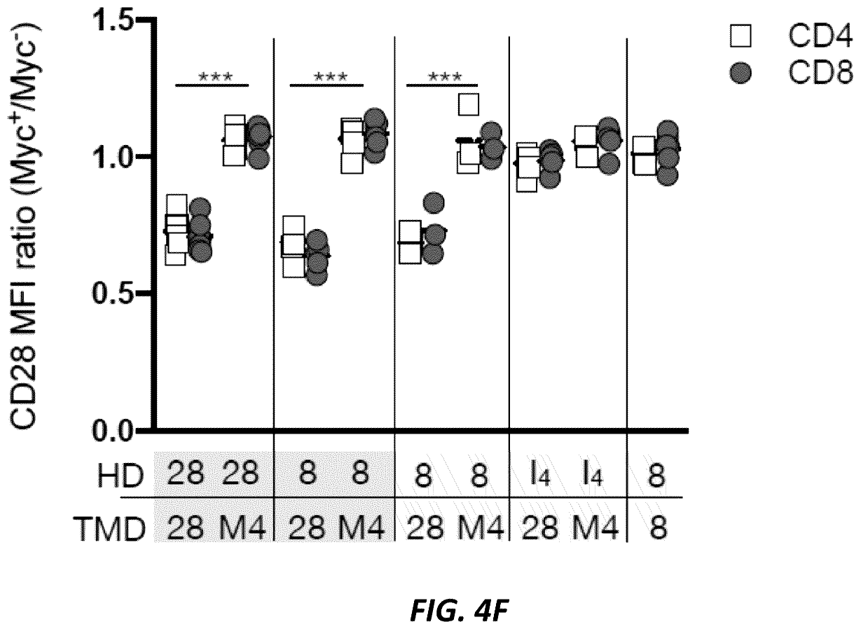

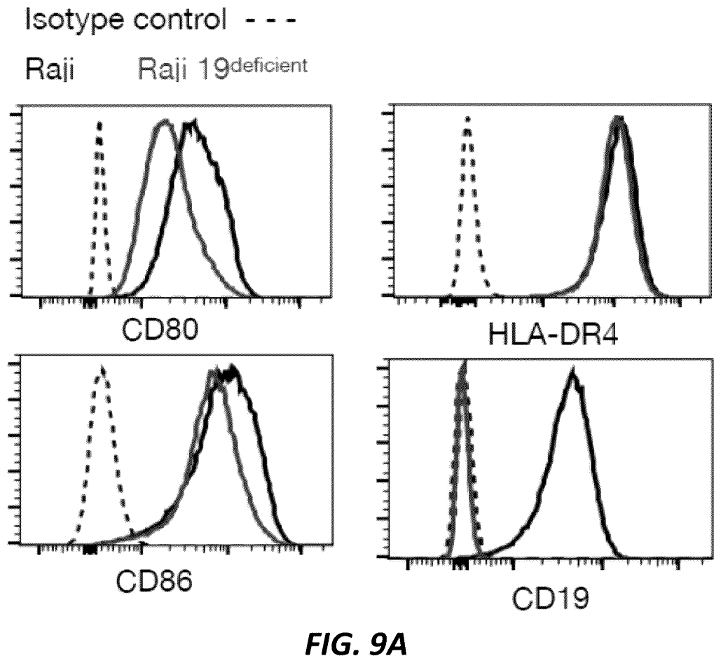

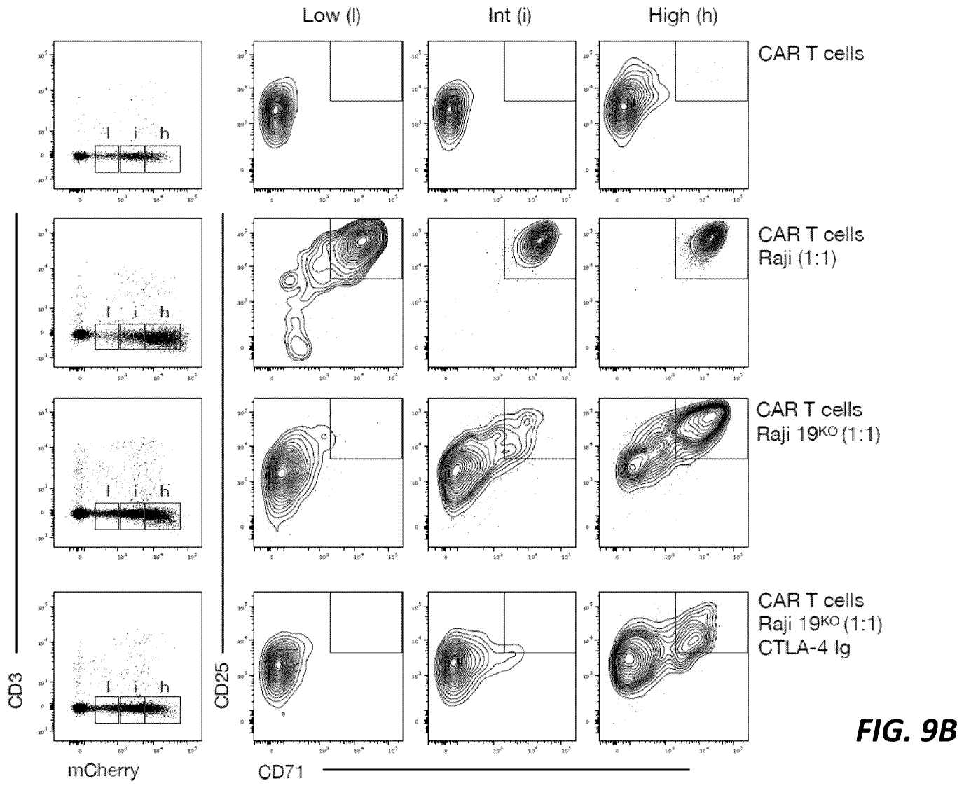

To determine if the natural ligands of CD28 can activate CARs by engaging CD28-CAR heterodimers, CAR T cells were stimulated with different HD and TMD with a mutant Raji lymphoma cell line that had the CD19 gene deleted using CRISPR/Cas9. It was observed that CD19-deficient Raji cells retained high levels of CD80 and CD86 expression (see, e.g., FIG. 9A) and induced CAR T cell activation, as measured by the upregulation of CD25 and CD71 expression (see, e.g., FIG. 4A and FIG. 9B). This activation was significantly reduced by CTLA-4 Ig, a high-affinity competitive inhibitor of CD28 by binding to CD80 and CD86 (FIGS. 4A-4B). This result indicates that the activation of CAR T cells is predominantly driven by the CD28 interaction with CD80 and CD86. This CD80/86-induced “off-target” activation was seen disproportionately in CAR T cells with high CAR expression (FIGS. 4A-4B). CD28^{M4}-TMD did not markedly affect the off-target activation of the IgG4-HD CAR, likely because of the weak heterodimerization between IgG4-HD/CD28^{WT}-TMD CAR and CD28 (see, e.g., FIG. 2C). Intriguingly, the CD28^{M4}-TMD significantly potentiated off-target activation of CD8-HD containing CAR T cells (see, e.g., FIG. 4B). Thus, off-target activation of CARs mediated by CD80/86 was not dependent on the heterodimerization between CD28 and CAR and may even be inhibited by the CD28-CAR heterodimerization. These results suggest that CD80/86-induced CAR activation is mediated through CD28 homodimers. With high CAR expression, CD80/86 may induce CAR clustering through membrane compartmentalization and off-target CAR activation as observed with CD3/CD28 beads (see, e.g., FIG. 3C). Altogether, without being bound to any particular theory, it was hypothesized that, in the context of a CD8-HD, the CD28 monomer associated with the CAR cannot efficiently engage its natural ligands CD80 and CD86, but still retains binding to anti-CD28, thus explaining the distinct results between CD19-deficient Raji and anti-CD28 stimulation.

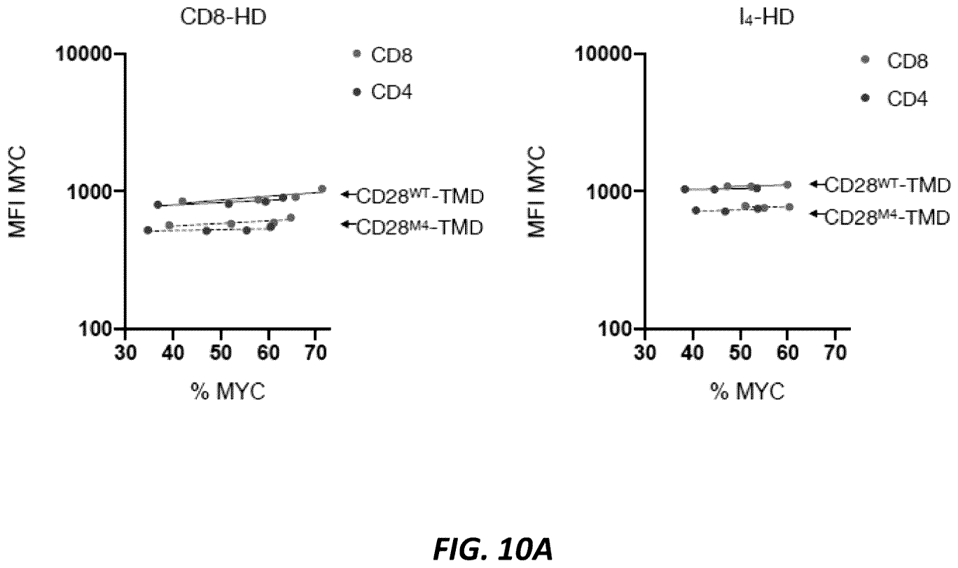

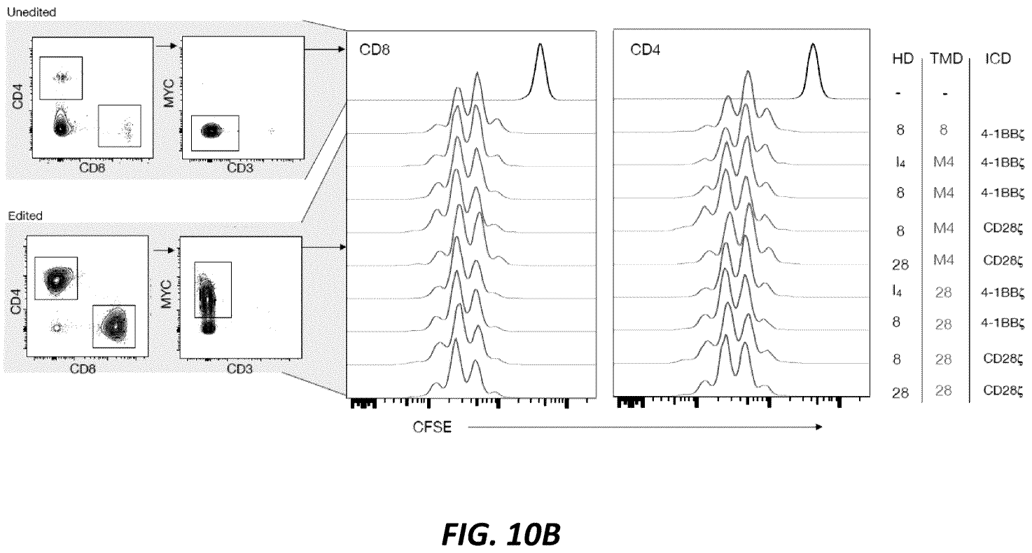

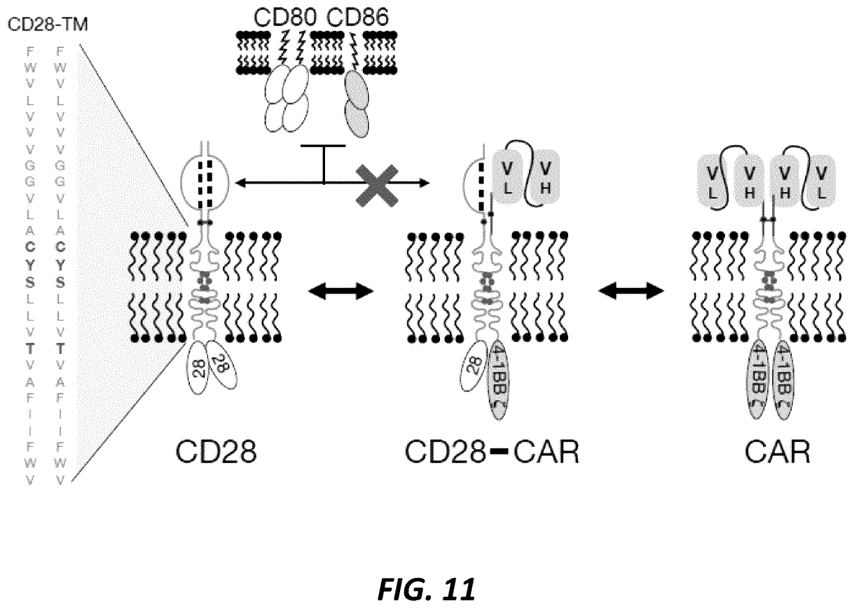

A previous report has shown that the percentages of CD28⁺ CD19-CAR T cells engineered with a CD28-TMD are significantly lower when compared to CD19-CAR T cells engineered with a CD8-TMD. This correlated with a significantly lower number of CD28-TMD-containing CAR T cells in the peripheral blood one month after infusion into patients, possibly suggesting that the reduced CD28 expression might have impaired CAR T cell persistence. Thus, it was hypothesized that CAR-CD28 heterodimerization could regulate CD28 expression. Since CAR expression levels greatly influence T cell phenotype and activation, various CARs were engineered by knocking them into the TCR alpha constant (TRAC) gene locus using homology-directed repair (see, e.g., FIG. 4C). Knock-in efficiencies ranged between 17-72% across the various CAR constructs. As previously demonstrated, this strategy resulted in a homogenous expression of the CAR independently of the percentage of editing (see, e.g., FIG. 10A). All CAR T cells proliferated upon stimulation with CD19⁺ NALM-6 target cells (see, e.g., FIG. 10B). Six days after CAR knock-in, it was observed a 26-51% reduction in CD28 expression of CAR T cells containing a CD28^{WT}-TMD as compared to a CD28^{M4}-TMD with either a CD8-HD or CD28-HD (see, e.g., FIGS. 4D-4F). This reduction was seen in both CD4 and CD8 T cells with CARs containing either 28ζ or 4-1BBζ ICD. The downregulation of CD28 in CAR T cells was only minimal with CAR T cells engineered with an IgG4-HD/CD28^{WT}-TMD, echoing with previously reported result of inefficient CAR-CD28 heterodimerization in the context of IgG4-HD. CD28 down-modulation occurred days after CAR engineering, independent of target antigen or CD28-mediated co-stimulation, suggesting that this might be mediated at the posttranscriptional level, most likely at the cell surface.

Taken together, the experimental data presented herein demonstrate that the CD28-TMD mediates CAR and CD28 heterodimerization via a core of four amino acids. The CAR-CD28 heterodimer triggers CAR T-cell activation after anti-CD28 stimulation independently of TCR and CAR cognate antigens but did not respond to natural CD28 ligands, CD80 and CD86. Yet, CAR-CD28 heterodimerization is associated with reduced cell surface expression of CD28. Together, these results demonstrate an active influence of the TMD and the HD on CAR T cells. Future investigations are needed to understand the contribution of the CAR-CD28 heterodimerization to CAR T cell activation, survival, exhaustion, and CAR T-cell associated toxicities. Thus, the experimental data presented herein indicate that CAR TMDs may modulate CAR T activities by association with their endogenous partners. Optimization of CAR designs should incorporate consideration of TMD-mediated receptor interactions.

### Example 5

### Modeling of Hinge-Hinge Interactions

This Example describes the results of experiments performed to model the hinge-hinge interactions, which is then used to investigate how the hinge domain (HD) influences the interaction of CD28 with CARs, given the impact of the HD on CAR-CD28 heterodimerization.

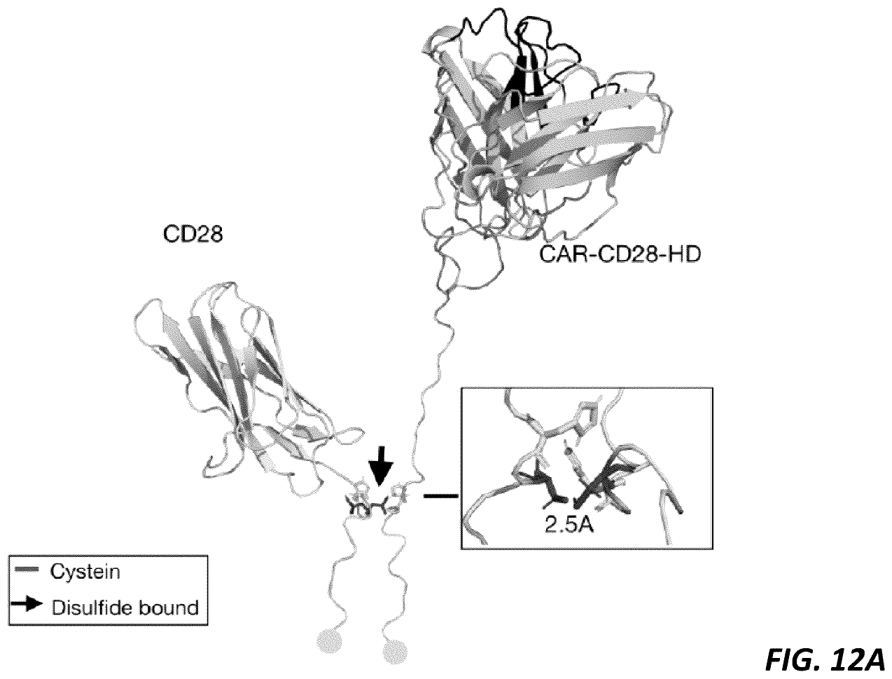

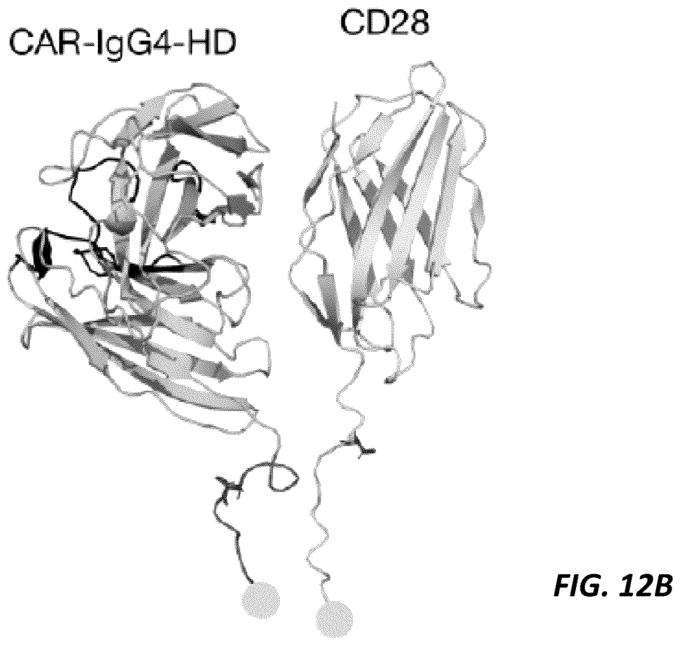

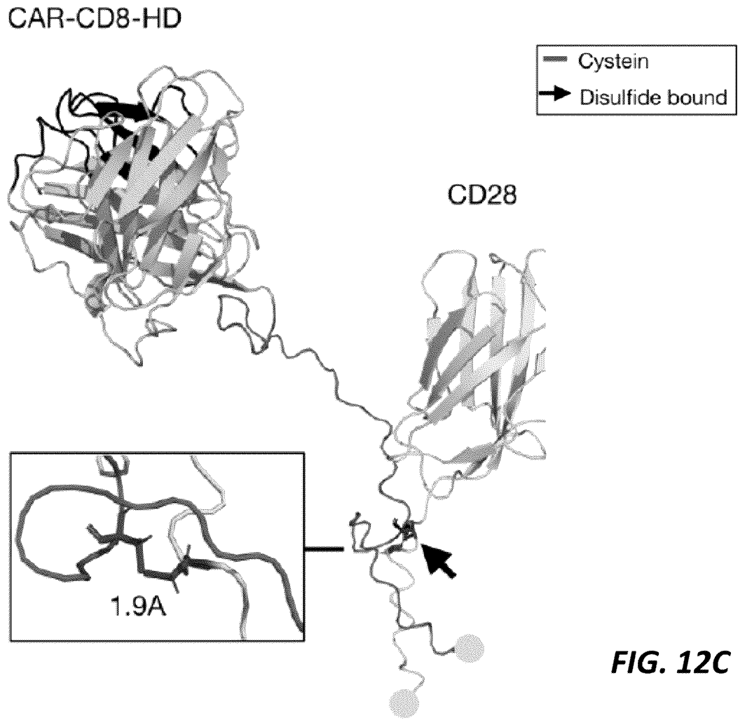

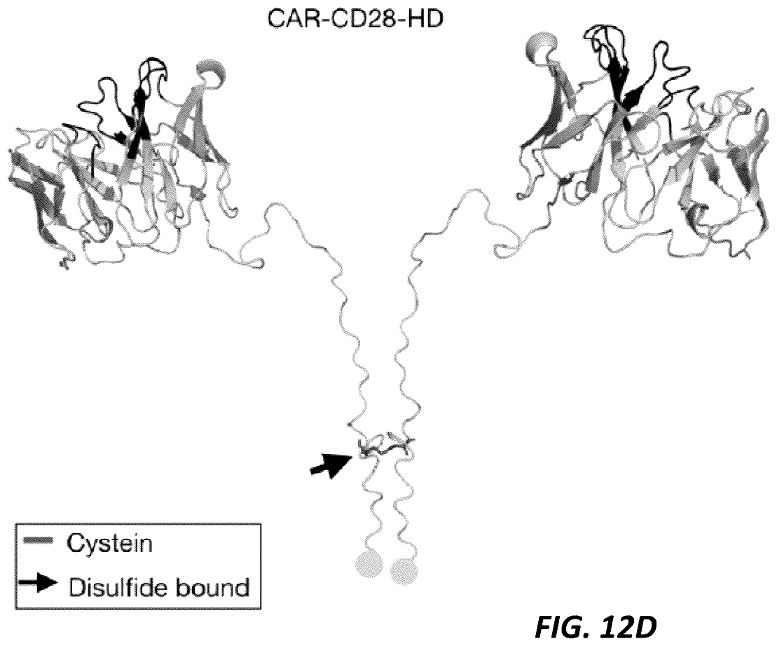

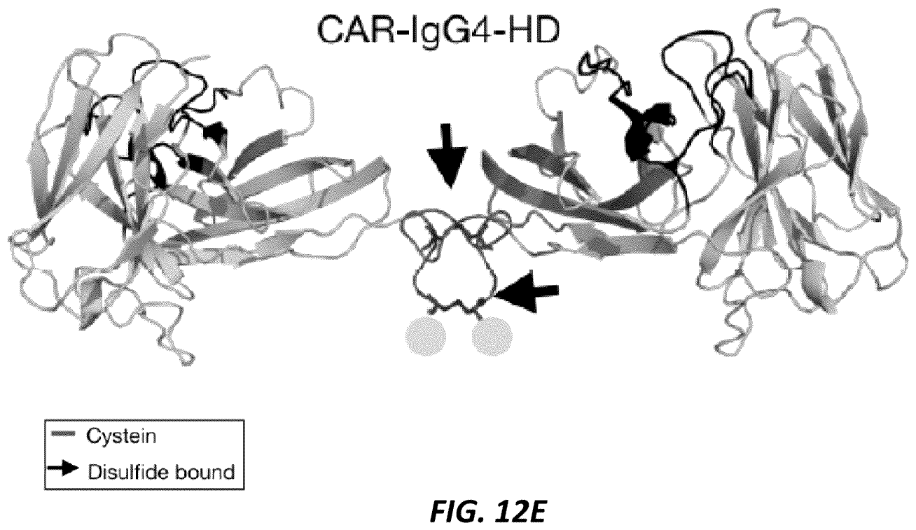

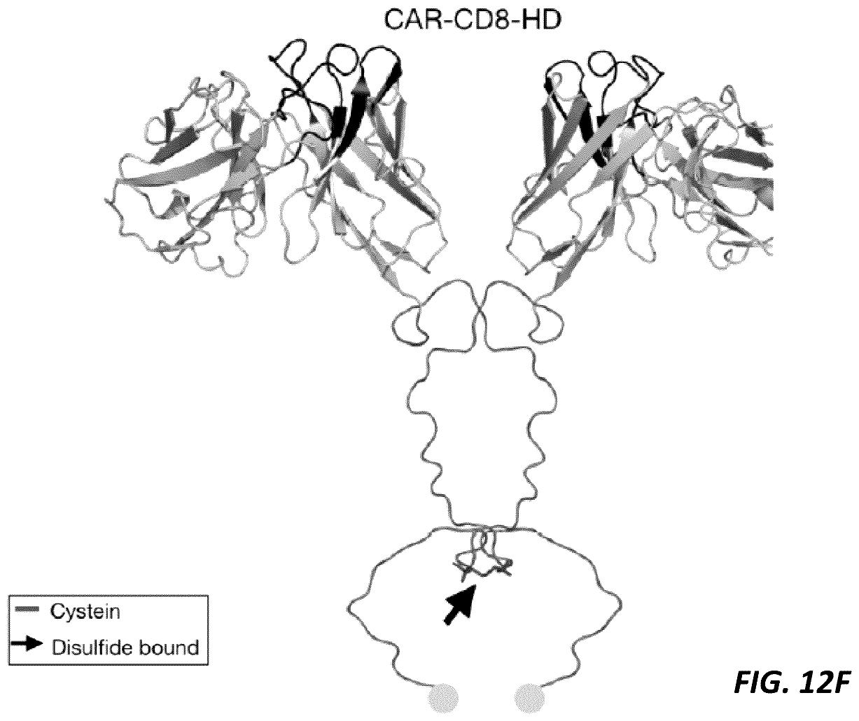

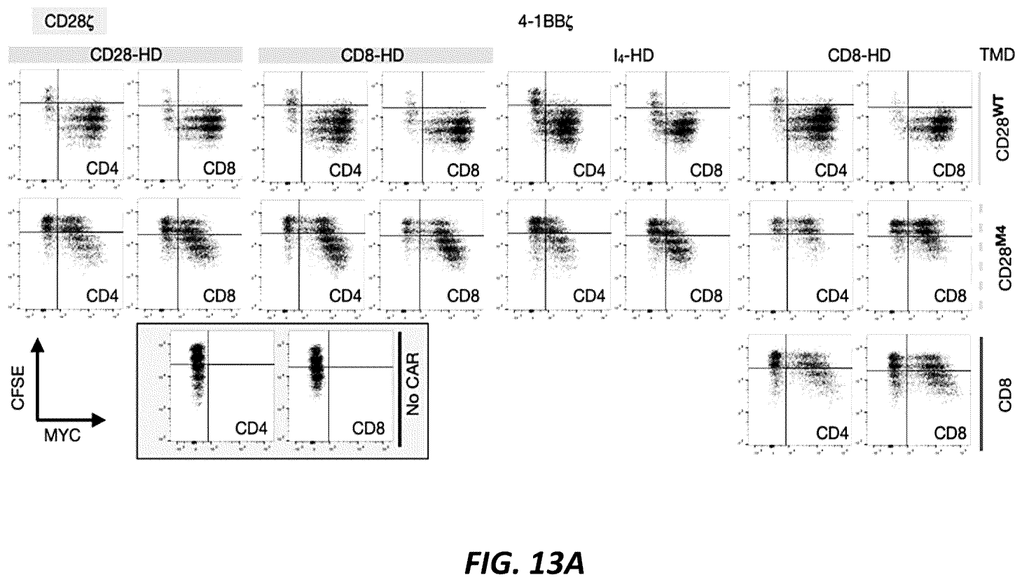

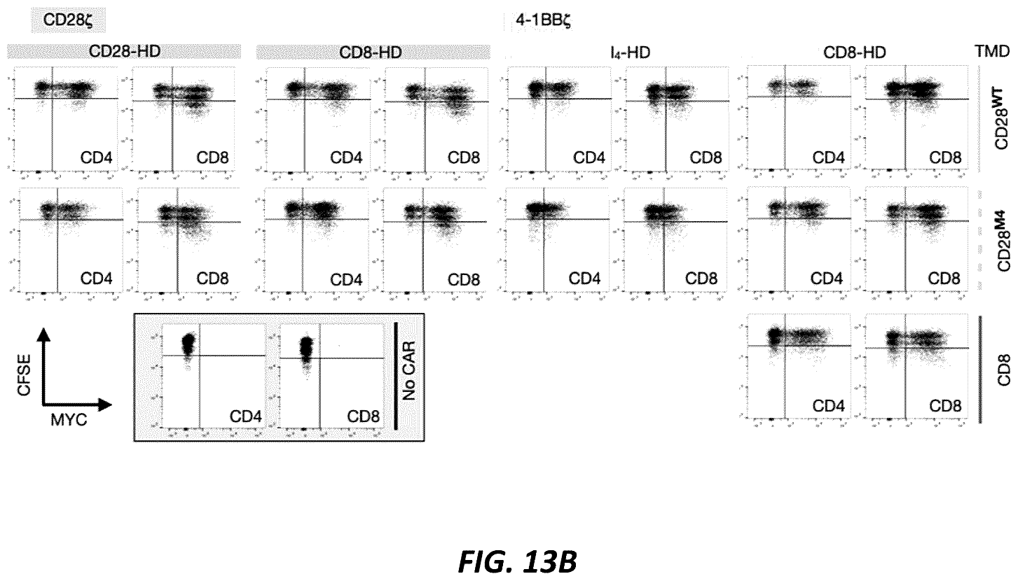

In these experiments, structural modeling of the different CARs was performed with the extracellular domain of the CAR and CD28 receptors, was performed using Iterative threading ASSembly Refinement (I-TASSER) software as described in Example 1. The cysteine residue in the HD of the CD28 receptor at position C123 (Leddon A S et al., Front Immunol. 11:1519, 2020) was aligned with the cysteine in the HD of CD28-HD-containing and CD8-HD-containing CARs (FIGS. 12A-12F). It was found that, for IgG4-HD-containing CARs, the cysteines in the HD could not be aligned with C123 of the CD28 (FIGS. 12A-12F). The modeling presented herein suggested that a disulfide bound between the endogenous CD28-HD and the CD28-HD- and CD8-HD-containing CARs is possible. However, as shown in FIGS. 4D-4F, the lack of CD28 downregulation in CAR T cells with a CD28-HD and a M4-CD28-TMD suggests the CD28-HD alone was not sufficient to drive the heterodimerization. Moreover, when various CD3− CAR+ T cells were stimulated with anti-CD3/CD28 beads, preferential CFSE dilution of T cells engineered with the CD28-HD/M4-CD28-TMD CAR construct was not observed when compared to CAR T cells with the CD8-HD/M4-CD28-TMD constructs, supporting the notion that the cysteine bridge in the CD28-HD is insufficient to mediate CD28-CAR heterodimerization (see, e.g., FIGS. 13A-13B). These results further support the notion that the cysteine bridge in the CD28-HD is insufficient to mediate CD28-CAR heterodimerization without interactions in the CD28-TMD. Taken together, these results demonstrate that cysteines and inter-molecular disulfide bonds in the HDs are not the drivers of CAR-CD28 heterodimerization but can also be involved in the stabilization of the CAR-CD28 heterodimers.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1. June, C H, Sadelain, M. Chimeric Antigen Receptor Therapy. N Engl J Med. 2018; 379(1):64-73.
2. Schuster, S J et al. Tisagenlecleucel in Adult Relapsed or Refractory Diffuse Large B Cell Lymphoma. N Engl J Med. 2019; 380(1):45-56.
3. Gardner, R A et al. Intent-to-treat leukemia remission by CD19 CAR T cells of defined formulation and dose in children and young adults. Blood. 2017; 129(25):3322-3331.
4. Locke, F L et al. Long-term safety and activity of axicabtagene ciloleucel in refractory large B-cell lymphoma (ZUMA-1): a single-arm, multicentre, phase 1-2 trial. Lancet Oncol. 2019; 20(1):31-42.
5. Bridgeman, J S, Hawkins, R E, Bagley, S, Blaylock, M, Holland, M, Gilham, D E. The optimal antigen response of chimeric antigen receptors harboring the CD3zeta transmembrane domain is dependent upon incorporation of the receptor into the endogenous TCR/CD3 complex. J Immunol. 2010; 184(12):6938-6949.
6. Frigault, M J et al. Identification of chimeric antigen receptors that mediate constitutive or inducible proliferation of T cells. Cancer Immunol Res. 2015; 3(4):356-367.
7. Majzner, R G et al. Tuning the Antigen Density Requirement for CAR T Cell Activity. Cancer Discov. 2020.
8. Alabanza, L et al. Function of Novel Anti-CD19 Chimeric Antigen Receptors with Human Variable Regions Is Affected by Hinge and Transmembrane Domains. Mol Ther. 2017; 25(11):2452-2465.
9. Kochenderfer, J N et al. Chemotherapy-refractory diffuse large B-cell lymphoma and indolent B-cell malignancies can be effectively treated with autologous T cells expressing an anti-CD19 chimeric antigen receptor. J Clin Oncol. 2015; 33(6):540-549.
10. Brudno, J N et al. Safety and feasibility of anti-CD19 CAR T cells with fully human binding domains in patients with B-cell lymphoma. Nat Med. 2020; 26(2):270-280.
11. Fink, A, Sal-Man, N, Gerber, D, Shai, Y. Transmembrane domains interactions within the membrane milieu: principles, advances and challenges. Biochim Biophys Acta. 2012; 1818(4):974-983.
12. Salter, A I et al. Phosphoproteomic analysis of chimeric antigen receptor signaling reveals kinetic and quantitative differences that affect cell function. Sci Signal. 2018; 11(544).
13. Ramello, M C et al. An immunoproteomic approach to characterize the CAR interactome and signalosome. Sci Signal. 2019; 12(568).
14. Aalberse, R C, Schuurman, J. IgG4 breaking the rules. Immunology. 2002; 105(1):9-19.
15. Khadria, A S, Mueller, B K, Stefely, J A, Tan, C H, Pagliarini, D J, Senes, A. A Gly-zipper motif mediates homodimerization of the transmembrane domain of the mitochondrial kinase ADCK3. J Am Chem Soc. 2014; 136(40):14068-14077.
16. Khan, A A, Bose, C, Yam, L S, Soloski, M J, Rupp, F. Physiological regulation of the immunological synapse by agrin. Science. 2001; 292(5522):1681-1686.
17. Lazar-Molnar, E, Almo, S C, Nathenson, S G. The interchain disulfide linkage is not a prerequisite but enhances CD28 costimulatory function. Cell Immunol. 2006; 244(2):125-129.
18. Eyquem, J et al. Targeting a CAR to the TRAC locus with CRISPR/Cas9 enhances tumour rejection. Nature. 2017; 543(7643):113-117.
19. Hildebrand, P W, Gunther, S, Goede, A, Forrest, L, Frommel, C, Preissner, R. Hydrogen-bonding and packing features of membrane proteins: functional implications. Biophys J. 2008; 94(6):1945-1953.
20. Roth, T L et al. Reprogramming human T cell function and specificity with non-viral genome targeting. Nature. 2018; 559(7714):405-409.
21. Hill, Z B, Martinko, A J, Nguyen, D P, Wells, J A. Human antibody-based chemically induced dimerizers for cell therapeutic applications. Nat Chem Biol. 2018; 14(2):112-117.
22. Rodgers, D T et al. Switch-mediated activation and retargeting of CAR-T cells for B-cell malignancies. Proc Natl Acad Sci USA. 2016; 113(4):E459-68.
23. Krogh, A, Larsson, B, von Heijne, G, Sonnhammer, E L. Predicting transmembrane protein topology with a hidden Markov model: application to complete genomes. J Mol Biol. 2001; 305(3):567-580.
24. Lee D W, Kochenderfer J N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet. (2015) 385:517-28. doi: 10.1016/S0140-6736(14)61403-3.
25. Porter D L, Hwang W T, Frey N V, Lacey S F, Shaw P A, Loren A W, et al. Chimeric antigen receptor T cells persist and induce sustained remissions in relapsed refractory chronic lymphocytic leukemia. Sci Transl Med. (2015) 7:303ra139. doi: 10.1126/scitranslmed.aac5415.
26. Maude S L, Laetsch T W, Buechner J, Rives S, Boyer M, Bittencourt H, et al. Tisagenlecleucel in children and young adults with B-cell lymphoblastic leukemia. Engl J Med. (2018) 378:439-48. doi: 10.1056/NEJMoa1709866.
27. Schuster S J, Svoboda J, Chong E A, Nasta S D, Mato A R, AnakÖ, et al. Chimeric antigen receptor T cells in refractory B-cell lymphomas. N Engl J Med. (2017) 377:2545-54. doi: 10.1056/NEJMoa1708566.
28. Yang J, Yan R, Roy A, Xu D, Poisson J, Zhang Y. The I-TASSER suite: protein structure and function prediction. Nat Methods. (2015) 12:7-8. doi: 10.1038/nmeth.3213.
29. Arnett K L, Harrison S C, Wiley D C. Crystal structure of a human CD3-epsilon/delta dimer in complex with a UCHT1 single-chain antibody fragment. Proc Natl Acad Sci USA. (2004) 101:16268-73. doi: 10.1073/pnas.0407359101.
30. Scapin G, Yang X, Prosise W W, McCoy M, Reichert P, Johnston J M, et al. Structure of full-length human anti-PD1 therapeutic IgG4 antibody pembrolizumab. Nat Struct Mol Biol. (2015) 22:953-8. doi: 10.1038/nsmb.3129.
31. Evans E J, Esnouf R M, Manso-Sancho R, Gilbert R J, James J R, Yu C, et al. Crystal structure of a soluble CD28-Fab complex. Nat Immunol. (2005) 6:271-9. doi: 10.1038/ni1170.
32. Leman J K, Weitzner B D, Lewis S M, Adolf-Bryfogle J, Alam N, Alford R F, et al. Macromolecular modeling and design in Rosetta: recent methods and frameworks. Nat Methods. (2020) 17:665-80. doi: 10.1038/s41592-020-0848-2.
33. Williams C J, Headd J J, Moriarty N W, Prisant M G, Videau L L, Deis L N, et al. MolProbity: more and better reference data for improved all-atom structure validation. Protein Sci. (2018) 27:293-315. doi: 10.1002/pro.3330.
34. Leddon S A, Fettis M M, Abramo K, Kelly R, Oleksyn D, Miller J. The CD28 transmembrane domain contains an essential dimerization motif. Front Immunol. (2020) 11:1519. doi: 10.3389/fimmu.2020.01519.
35. Call M E, Schnell J R, Xu C, Lutz R A, Chou J J, Wucherpfennig K W. The structure of the zetazeta transmembrane dimer reveals features essential for its assembly with the T cell receptor. Cell. (2006) 127:355-68. doi: 10.1016/j.cell.2006.08.044.
36. Feucht J, Sun J, Eyquem J, Ho Y J, Zhao Z, Leibold J, et al. Calibration of CAR activation potential directs alternative T cell fates and therapeutic potency. Nat Med. (2019) 25:82-8. doi: 10.1038/s41591-018-0290-5.
37. Park J H, Rivière I, Gonen M, Wang X, Sénéchal B, Curran K J, et al. Long-term follow-up of CD19 CAR therapy in acute lymphoblastic leukemia. N Engl J Med. (2018) 378:449-59. doi: 10.1056/NEJMoa1709919.
38. Ying Z, He T, Wang X, Zheng W, Lin N, Tu M, et al. Parallel comparison of 4-1BB or CD28 co-stimulated CD19-targeted CAR-T cells for B cell Non-Hodgkin's lymphoma. Mol Ther Oncolytics. (2019) 15:60-8. doi: 10.1016/j.omto.2019.08.002.
39. Santomasso B D, Park J H, Salloum D, Riviere I, Flynn J, Mead E, et al. Clinical and biological correlates of neurotoxicity associated with CAR T-cell therapy in patients with B-cell acute lymphoblastic leukemia. Cancer Discov. (2018) 8:958-71. doi: 10.1158/2159-8290.CD-17-1319.
40. Maziarz R T, Schuster S J, Romanov V V, Rusch E S, Li J, Signorovitch J E, et al. Grading of neurological toxicity in patients treated with tisagenlecleucel in the JULIET trial. Blood Adv. (2020) 4:1440-7. doi: 10.1182/bloodadvances.2019001305.
41. Parker K R, Migliorini D, Perkey E, Yost K E, Bhaduri A, Bagga P, et al. Single-cell analyses identify brain mural cells expressing CD19 as potential off-tumor targets for CAR-T immunotherapies. Cell. (2020) 183:126-42.e17. doi: 10.1016/j.cell.2020.08.022.

What is claimed is:

1. A chimeric polypeptide comprising:
(a) an extracellular domain (ECD) having a binding affinity for an antigen;
(b) a hinge domain;
(c) a CD28 transmembrane domain (TMD); and
(d) an intracellular signaling domain (ICD).

2. The chimeric polypeptide of claim 1, wherein the CD28-TMD is a mouse CD28-TMD or a human CD28-TMD.

3. The chimeric polypeptide of any one of claims 1 to 2, wherein the TMD comprises one or more amino acid substitutions within a transmembrane dimerization motif of the CD28-TMD.

4. The chimeric polypeptide of any one of claims 1 to 3, wherein the TMD comprises an amino acid sequence having at least 70% sequence identity to a CD28-TMD having the sequence of SEQ ID NO: 1, and further comprises one or more amino acid substitutions at a position corresponding to an amino acid residue selected from the group consisting of X13, X14, X15, and X19 of SEQ ID NO: 1.

5. The chimeric polypeptide of any one of claims 1 to 3, wherein the TMD comprises the sequence of SEQ ID NO: 1, and further comprises one or more amino acid substitutions at an amino acid residue selected from the group consisting of C13, Y14, S15, and T19 of SEQ ID NO: 1.

6. The chimeric polypeptide of any one of claims 1 to 5, wherein the TMD comprises the sequence of SEQ ID NO: 6.

7. The chimeric polypeptide of any one of claims 1 to 6, wherein the TMD comprises the sequence of SEQ ID NO: 6, and wherein one, two, three, four, or five amino acid residues in the sequence of SEQ ID NO: 6 is optionally substituted by a different amino acid residue.

8. The chimeric polypeptide of any one of claims 3 to 7, wherein the one or more amino acid substitutions is independently selected from the group consisting of a leucine substitution, an alanine substitution, an arginine substitution, an aspartic acid substitution, a histidine substitution, a glutamic acid substitution, a lysine substitution, a serine substitution, a tryptophan substitution, and combinations of any thereof.

9. The chimeric polypeptide of any one of claims 3 to 8, wherein at least one of the one or more amino acid substitutions is a nonpolar-to-polar amino acid substitution.

10. The method of any one of claims 3 to 9, wherein the one or more amino acid substitutions results in reduced binding of the chimeric polypeptide to a CD28 polypeptide compared to a chimeric polypeptide comprising a CD28-TMD that lacks the one or more amino acid substitution.

11. The chimeric polypeptide of any one of claims 1 to 10, wherein the chimeric polypeptide is a chimeric antigen receptor (CAR).

12. The chimeric polypeptide of any one of claims 1 to 11, wherein the hinge domain is derived from a CD8α hinge domain, a CD28 hinge domain, an IgG4 hinge domain, and an Ig4 CH2-CH3 domain.

13. The chimeric polypeptide of any one of claims 1 to 12, wherein the ICD comprises one or more costimulatory domains selected from the group consisting of costimulatory domains derived from 4-1BB (CD137), CD27 (TNFRSF7), CD28, CD70, LFA-2 (CD2), CD5, ICAM-1 (CD54), ICOS, LFA-1 (CD11a/CD18), DAP10, and DAP12.

14. The chimeric polypeptide of claim 13, wherein the ICD comprises two costimulatory domains.

15. The chimeric polypeptide of any one of claims 1 to 14, wherein the ECD comprises an antigen-binding moiety capable of binding to an antigen on the surface of a cell.

16. The chimeric antigen of claim 15, wherein the antigen-binding moiety is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment of any thereof.

17. The method of claim 16, wherein the antigen-binding moiety comprises a scFv.

18. The method of any one of claims 1 to 17, wherein the antigen is a tumor associated-antigen or a tumor-specific antigen.

19. The chimeric polypeptide of claim 18, wherein the antigen is selected from the group consisting of CD19 and HLA-A2.

20. The chimeric polypeptide of any one of claims 1 to 19, wherein the ICD further comprises a CD3ζ domain.

21. The chimeric polypeptide of claim 20, wherein the CD3ζ domain comprises the sequence of SEQ ID NO: 17 or SEQ ID NO: 18, or a functional variant thereof.

22. A nucleic acid construct comprising a nucleic acid sequence that encodes a chimeric polypeptide according to of any one of claims 1 to 21.

23. The nucleic acid construct of claim 22, wherein the nucleotide sequence is incorporated into an expression cassette or an expression vector.

24. The nucleic acid construct of claim 23, wherein the expression vector is a viral vector.

25. The nucleic acid construct of claim 24, wherein the viral vector is a lentiviral vector, an adeno virus vector, an adeno-associated virus vector, or a retroviral vector.

26. A recombinant cell comprising:
a chimeric polypeptide according to any one of claims 1 to 21; and/or
a nucleic acid according to any one of claims 22 to 25;

27. The recombinant cell of claim 26, wherein the recombinant cell is a eukaryotic cell.

28. The recombinant cell of any one of claim 26 to 27, wherein the recombinant cell is an immune system cell.

29. The recombinant cell of claim 28, wherein the immune system cell is a T lymphocyte.

30. A cell culture comprising at least one recombinant cell according to any one of claims 26 to 29 and a culture medium.

31. A pharmaceutical composition comprising a pharmaceutically acceptable excipient and one or more of the following:
a) a chimeric polypeptide according to any one of any one of claims 1 to 21;
b) a nucleic acid construct according to any one of claims 22 to 25.
c) a recombinant cell according to any one of claims 26 to 29.

32. The pharmaceutical composition of claim 31, wherein the composition comprises a recombinant cell according to any one of claims 26 to 29, and a pharmaceutically acceptable excipient.

33. The pharmaceutical composition of claim 31, wherein the composition comprises a nucleic acid construct according to any one of claims 22 to 25, and a pharmaceutically acceptable excipient.

34. The pharmaceutical composition of claim 33, wherein the nucleic acid construct is encapsulated in a viral capsid or a lipid nanoparticle.

35. A method for modulating T-cell activation in a subject having or suspected of having a health condition, comprising administering to the subject a composition comprising at least one recombinant cell according to any one of claims 26 to 29; and/or a pharmaceutical composition according to any one of claims 31 to 34.

36. A method for treating a health condition in a subject in need thereof, comprising administering to the subject a composition comprising at least one recombinant cell according to any one of claims 26 to 29; and/or a pharmaceutical composition according to any one of claims 31 to 34.

37. The method of any one of claims 35 to 36, wherein the health condition is a proliferative disorder, an autoimmune disorder, or an infection.

38. The method of claim 37, wherein the proliferative disorder is a cancer.

39. The method of any one of claims 37 to 38, wherein the proliferative disorder is a cancer selected from the group consisting of a lymphoma, acute lymphocytic leukemia, and relapsed/refractory large B-cell lymphoma.

40. The method of claim 39, wherein the lymphoma is a Burkitt lymphoma.

41. The method of any one of claims 36 to 40, wherein the administered composition results in reduced on-target activation in the subject.

42. The method of any one of claims 36 to 40, wherein the administered composition increases CAR-T cell survival in the subject.

43. The method of any one of claims 36 to 40, wherein the administered composition reduces CAR-T cell exhaustion in the subject.

44. The method of any one of claims 36 to 40, wherein the administered composition reduces CAR-T cell toxicity in the subject.

45. The method of any one of claims 36 to 40, wherein the administered composition inhibits tumor growth or metastasis of a cancer in the subject.

46. The method of any one of claims 36 to 45, wherein the composition is administered to the subject individually (monotherapy) or as a first therapy in combination with a second therapy (multi-therapy).

47. The method of claim 46, wherein the second therapy is selected from the group consisting of chemotherapy, radiotherapy, immunotherapy, hormonal therapy, toxin therapy, or surgery.

48. The method of any one of claims 46 to 47, wherein the first therapy and the second therapy are administered concomitantly.

49. The method of any one of claims 46 to 48, wherein the first therapy is administered at the same time as the second therapy.

50. The method of any one of claims 46 to 47, wherein the first therapy and the second therapy are administered sequentially.

51. The method of claim 50, wherein the first therapy is administered before the second therapy.

52. The method of claim 50, wherein the first therapy is administered after the second therapy.

53. The method of any one of claims 46 to 47, wherein the first therapy is administered before and/or after the second therapy.

54. The method of any one of claims 46 to 47, wherein the first therapy and the second therapy are administered in rotation.

55. The method of any one of claims 46 to 47, wherein the first therapy and the second therapy are administered together in a single formulation.

56. A kit for modulating T-cell activation in a subject, or for treating a health condition in a subject in need thereof, the kit comprising instructions for use thereof and one or more of the following:
a) one or more chimeric polypeptides according to any one of any one of claims 1 to 21;
b) one or more nucleic acid constructs according to any one of claims 22 to 25;
c) one or more recombinant cells according to any one of claims 26 to 29; and
d) one or more pharmaceutical compositions according to any one of claims 31 to 34.


SEQUENCE LISTING (25 sequences)

SEQ ID NO: 1 - protein, 27 aa, Artificial sequence
  Synthetic construct
  MISC_FEATURE: Human CD28-TMD
FWVLVVVGGVLACYSLLVTVAFIIFWV

SEQ ID NO: 2 - protein, 27 aa, Artificial sequence
  Synthetic construct
  MISC_FEATURE: Mouse CD28-TMD
FWALVVVAGVLFCYGLLVTVALCVIWT

SEQ ID NO: 3 - protein, 27 aa, Artificial sequence
  Synthetic construct
  MISC_FEATURE: M1 mutein of CD28-TMD
FWVLVVVLLVLACYSLLVTVAFIIFWV

SEQ ID NO: 4 - protein, 27 aa, Artificial sequence
  Synthetic construct
  MISC_FEATURE: M2 mutein of CD28-TMD
FWVLVVVGGVLAAYSLLVTVAFIIFWV

SEQ ID NO: 5 - protein, 27 aa, Artificial sequence
  Synthetic construct
  MISC_FEATURE: M3 mutein of CD28-TMD
FLVLVVVGGVLACYSLLVTVAFIIFLV

SEQ ID NO: 6 - protein, 27 aa, Artificial sequence
  Synthetic construct
  MISC_FEATURE: M4 mutein of CD28-TMD
FWVLVVVGGVLALLLLLVLVAFIIFWV

SEQ ID NO: 7 - DNA, 19 bp, Artificial sequence
  Synthetic construct
  MISC_FEATURE: Guide RNA for TRBC
ccaccagctcagctccacg

SEQ ID NO: 8 - DNA, 20 bp, Artificial sequence
  Synthetic construct
  MISC_FEATURE: Guide RNA for CD19
cgaggaacctctagtggtga

SEQ ID NO: 9 - DNA, 20 bp, Artificial sequence
  Synthetic construct
  MISC_FEATURE: Guide RNA for CD28
ttcaggtttactcaaaaacg

SEQ ID NO: 10 - protein, 242 aa, Artificial sequence
  Synthetic construct
  MISC_FEATURE: CD19 CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 11 - protein, 13 aa, Artificial sequence
  Synthetic construct
  MISC_FEATURE: IgG4 hinge domain
ESKYGPPCPPCPM

SEQ ID NO: 12 - protein, 45 aa, Artificial sequence
  Synthetic construct
  MISC_FEATURE: CD8 Alpha hinge domain
TTTPAPRPPTPAPTIASQPLSLRPEACRPAAGGAVHTRGLDFACD

SEQ ID NO: 13 - protein, 39 aa, Artificial sequence
  Synthetic construct
  MISC_FEATURE: CD28 hinge domain
IEVMYPPPYLDNEKSNGTIIHVKGKHLCPSPLFPGPSKP

SEQ ID NO: 14 - protein, 24 aa, Artificial sequence
  Synthetic construct
  MISC_FEATURE: CD8 transmembrane domain
IYIWAPLAGTCGVLLLSLVITLYC

SEQ ID NO: 15 - protein, 42 aa, Artificial sequence
  Synthetic construct
  MISC_FEATURE: 4-1BB ICD (intracellular domain) Peptide
KRGRKKLLYIFKQPFMRPVQTTQEEDGCSCRFPEEEEGGCEL

SEQ ID NO: 16 - protein, 41 aa, Artificial sequence
  Synthetic construct
  MISC_FEATURE: CD28 ICD
RSKRSRLLHSDYMNMTPRRPGPTRKHYQPYAPPRDFAAYRS

SEQ ID NO: 17 - protein, 112 aa, Artificial sequence
  Synthetic construct
  MISC_FEATURE: Wild-type CD3 Zeta ICD
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 112 aa, Artificial sequence
  Synthetic construct
  MISC_FEATURE: CD3 Zeta ICD variant with 14Q-->K substitution
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 81 aa, Artificial sequence
  Synthetic construct
  MISC_FEATURE: IgG4 hinge - CD8 TMD
FLKMNSLQTDDTAIYYCAKHYYYGGSYAMDYWGQGTSVTVSSESKYGPPCPPCPMIWAPLAGTCGVLLLSLVITLYCKRGR

SEQ ID NO: 20 - protein, 81 aa, Artificial sequence
  Synthetic construct
  MISC_FEATURE: CD8 hinge - CD8 TMD
GTSVTVSSTTTPAPRPPTPAPTIASQPLSLRPEACRPAAGGAVHTRGLDFACDIYIWAPLAGTCGVLLLSLVITLYCKRGR

SEQ ID NO: 21 - protein, 81 aa, Artificial sequence
  Synthetic construct
  MISC_FEATURE: CD8 hinge - 28 TMD
VTVSSTTTPAPRPPTPAPTIASQPLSLRPEACRPAAGGAVHTRGLDFACDFWVLVVVGGVLACYSLLVTVAFIIFWVKRGR

SEQ ID NO: 22 - protein, 81 aa, Artificial sequence
  Synthetic construct
  MISC_FEATURE: CD28 hinge - 28 TMD
WGQGTSVTVSSIEVMYPPPYLDNEKSNGTIIHVKGKHLCPSPLFPGPSKPFWVLVVVGGVLACYSLLVTVAFIIFWVKRGR

SEQ ID NO: 23 - protein, 81 aa, Artificial sequence
  Synthetic construct
  MISC_FEATURE: IgG4 hinge - 28 TMD
SLQTDDTAIYYCAKHYYYGGSYAMDYWGQGTSVTVSSESKYGPPCPPCPMFWVLVVVGGVLACYSLLVTVAFIIFWVKRGR

SEQ ID NO: 24 - DNA, 19 bp, Artificial sequence
  Synthetic construct
  MISC_FEATURE: Guide RNA for the constant chain of the TCR alpha gene (TRAC)
cagggttctggatatctgt

SEQ ID NO: 25 - protein, 21 aa, Artificial sequence
  Synthetic construct
  MISC_FEATURE: Signal peptide of CD8 alpha
MALPVTALLLPLALLLHAARP